(19) United States
(12) Patent Application Publication
(10) Pub. No.: US 2024/0389528 A1
(43) Pub. Date: Nov. 28, 2024

(54) PERFORMANCE GAIN IN ALS INHIBITOR HERBICIDE TOLERANT BETA VULGARIS PLANTS BY COMBINATION OF BEST FITTING ALS LARGE AND SMALL SUBUNITS

(71) Applicants: BAYER AKTIENGESELLSCHAFT, Leverkusen (DE); KWS SAAT SE & CO. KGAA, Einbeck (DE)

(72) Inventors: Corinna STREITNER, Melle (DE); Olaf CZARNECKI, Berlin (DE); Maik GERTZ, Hannover (DE); Vinicius VILPERTE, Einbeck (DE); Erik WESTBERG, Einbeck (DE); Harald AIGNER, Einbeck (DE); Jens Christoph LEIN, Göttingen (DE)

(21) Appl. No.: 18/687,796

(22) PCT Filed: Aug. 29, 2022
(86) PCT No.: PCT/EP2022/073943

§ 371 Date: Feb. 28, 2024

(30) Foreign Application Priority Data
Sep. 2, 2021 (NZ) 779721

(51) Int. Cl. A01H 1/04; A01H 1/00; A01H 5/06; A01H 6/02

(52) U.S. Cl. CPC A01H 1/045; A01H 1/123; A01H 5/06; A01H 6/024

(57) ABSTRACT

Provided are improved herbicide tolerant Beta vulgaris, particularly improved sugar beet plants, with increased yield performance, while maintaining optimal and agronomically relevant herbicide tolerance, wherein the large and small subunits of the herbicide tolerance acetolactate synthase enzyme are optimally fitted. Further provided are markers for identifying such improved herbicide tolerant Beta vulgaris plants, as well methods for obtaining and identifying such improved herbicide tolerant Beta vulgaris plants.

### INCORPORATION OF SEQUENCE LISTING

The sequence listing that is contained in the filed named “BCS11027.xml” which contains 48 sequence entries, is 80 kilo bytes (measured in operating system MS-Windows) and which is filed herewith is incorporated herein by reference in its entirety.

### FIELD OF THE INVENTION

The present invention relates to herbicide tolerant Beta vulgaris plants, such as ALS inhibitor herbicide tolerant Beta vulgaris plants, particularly sugar beet plants, as well as to methods of controlling unwanted vegetation in areas of growing Beta vulgaris plants by applying ALS inhibitor herbicides to such plants.

In particular, the invention relates to Beta vulgaris plants comprising a herbicide tolerant ALS allele coding for the ALS large catalytic subunit, combined with optimal regulatory subunits (ALS small subunit) to increase the yield, sugar yield, performance and/or vigor of the resulting plants.

### BACKGROUND

Cultivated forms of Beta vulgaris (as defined in Ford-Lloyd (2005) Sources of genetic variation, Genus Beta. In: Biancardi E, Campbell L G, Skaracis G N, De Biaggi M (eds) Genetics and Breeding of Sugar Beet. Science Publishers, Enfield (NH), USA, pp 25-33) are important agricultural crops in temperate and subtropical regions. For example, about 20% of the world sugar production is based on sugar beet. Because beet seedlings and juvenile plants during their first 6-8 weeks of their life are susceptible to strong competition caused by fast growing weeds, which outcompete the young crop plants, reliable weed control measures are imperative in these crop areas.

Herbicides are convenient tools to control weeds in cultured beets. The products used for this purpose, like phenmedipham, desmediphan and metamitron allow to suppress the growth of weeds in beet fields without damaging the crop. Nevertheless, under adverse environmental conditions the efficacy of these products leaves room for improvements, especially if noxious weeds like Chenopodium album, Amaranthus retroflexus, Fallopia convolvulus and/or Tripleurospermum inodorata germinate over an extended period of time.

ALS inhibitor herbicides are widely used in modern agriculture due to their effectiveness at moderate application rates and relative non-toxicity in animals. By inhibiting ALS activity, these families of herbicides prevent further growth and development of susceptible plants including many weed species. In order to provide plants with an increased tolerance to even high concentrations of ALS inhibitor herbicides that may be required for sufficient weed control, additional ALS-inhibiting herbicide-resistant breeding lines and varieties of crop plants, as well as methods and compositions for the production and use of ALS inhibiting herbicide-resistant breeding lines and varieties, have been developed.

These ALS inhibitor herbicides inhibit the enzyme “acetohydroxyacid synthase” (AHAS), also known as “acetolactate synthase” (ALS [EC 4.1.3.18]). ALS is the site of action of five structurally diverse herbicide families belonging to the class of ALS inhibitor herbicides, like (a) sulfonylurea herbicides (Beyer E. M et al. (1988), Sulfonylureas in Herbicides: Chemistry, Degradation, and Mode of Action; Marcel Dekker, New York, 1988, 117-189), (b) sulfonylaminocarbonyltriazolinone herbicides (Pontzen, R., Pflanz.-Nachrichten Bayer, 2002, 55, 37-52), (c) imidazolinone herbicides (Shaner, D. L., et al., Plant Physiol., 1984, 76, 545-546; Shaner, D. L., and O'Connor, S. L. (Eds.) The Imidazolinone Herbicides, CRC Press, Boca Raton, FL, 1991), (d) triazolopyrimidine herbicides (Kleschick, W. A. et al., Agric. Food Chem., 1992, 40, 1083-1085), and (e) pyrimidinyl(thio)benzoate herbicides (Shimizu, T. J., Pestic. Sci., 1997, 22, 245-256; Shimizu, T. et al., Acetolactate Syntehase Inhibitors in Herbicide Classes in Development, Böger, P., Wakabayashi, K., Hirai, K., (Eds.), Springer Verlag, Berlin, 2002, 1-41).

ALS is involved in the conversion of two pyruvate molecules to an acetolactate molecule and carbon dioxide. The reaction uses thyamine pyrophosphate in order to link the two pyruvate molecules. The resulting product of this reaction, acetolactate, eventually becomes valine, leucine and isoleucine (Singh (1999) “Biosynthesis of valine, leucine and isoleucine”, in Plant Amino Acids, Singh, B. K., ed., Marcel Dekker Inc. New York, New York, pp. 227-247).

The ALS holoenzyme consists of 4 catalytical and 4 regulatory subunits (Duggleby et al., Plant Physiol Biochem, 2008, Structure and mechanism of inhibition of plant acetohydroxyacid synthase). While the catalytical part is built of identical subunits, the regulatory part can be assembled from different regulatory subunits (Binder, S. (2010). Branched-chain amino acid metabolism in Arabidopsis thaliana. The Arabidopsis Book/American Society of Plant Biologists, 8). The regulatory subunits have a role in mediating the feedback inhibition by the BCAA (branched chain amino acids) leucine, valine and isoleucine (Lee et al., Biochemistry, 2001, Identification of the regulatory subunit of Arabidopsis thaliana acetohydroxyacid synthase and reconstitution with its catalytic subunit; Lee et al., FEBS, 2002, Regulatory interactions in Arabidopsis thaliana acetohydroxyacid synthase).

Inhibitors of ALS interrupt the biosynthesis of valine, leucine and isoleucine in plants. The consequence is an immediate depletion of the respective amino acid pools causing a stop of protein biosynthesis leading to a cessation of plant growth and eventually the plant dies, or—at least—is damaged.

Single base pair substitutions at specific sites of the catalytical subunit of ALS can lead to more or less resistant ALS enzyme variants which show different levels of inhibition by the ALS inhibitor herbicides. Plants comprising mutant ALS large subunit alleles therefore show different levels of tolerance to ALS inhibitor herbicides, depending on the chemical structure of the ALS inhibitor herbicide and the site of the point mutation in the ALS gene.

WO2012049268A1 describes the identification and isolation in Beta vulgaris of an ALS variant allele comprising a substitution in codon at nucleotide positions 1705-1707 of the endogenous ALS gene thereby encoding an ALS polypeptide having leucine instead of the naturally occurring tryptophan at amino acid position 569 (hereinafter also referred to as BvALS_W569L gene or allele). Beta vulgaris plants comprising this allele exhibit a strong and agronomically relevant tolerance to various ALS inhibitor herbicides as described also in WO2012049266A1.

Herbicide-tolerance mutations can impact the ALS enzyme activity. Moreover herbicide-tolerance mutations in ALS can reduce feedback inhibition and lead to accumulation of BCAA (Endo et al., 2013). These potential negative effects can be balanced by counteracting modifications in the regulatory subunits.

Thus, there remains a need for Beta vulgaris plants, such as sugar beet plants or fodder beet plants, comprising an ALS herbicide tolerance catalytic subunit allele, such as BvALS_W569L, combined with optimal fitting regulatory ALS small subunits to yield improved Beta vulgaris plants with increased performance as expressed inter alia by increased yield, such as increased sugar yield, while maintaining optimal and agronomically relevant ALS herbicide tolerance. This problem is solved as described hereinafter in the description, drawings and claims.

### SUMMARY OF THE INVENTION

In a first aspect, the invention provides an acetolactate synthase (ALS) inhibitor-herbicide tolerant Beta vulgaris plant, or hybrid seed comprising an ALS holo-enzyme comprising a large subunit of ALS comprising an amino acid sequence having at least 95%, 96%, 97%, 98% or 99% sequence identity with the amino acid sequence of SEQ ID NO. 1 and further comprising a leucine at a position corresponding to amino acid position 569 instead of the naturally occurring tryptophan, such as a large subunit of ALS comprising the amino acid sequence of SEQ ID NO. 3 or being encoded by a nucleotide sequence comprises the nucleotide sequence of SEQ ID NO. 4; and a small subunit of ALS which can be selected by identification with marker M1 (comprising the nucleotide sequence of SEQ ID NO. 33), marker M2 (comprising the nucleotide sequence of SEQ ID NO. 34), marker M3 (comprising the nucleotide sequence of SEQ ID NO. 35) or marker M4 (comprising the nucleotide sequence of SEQ ID NO. 36). The small subunit of ALS may be encoded by a chromosomal region located on chromosome 3, between a marker selected from marker M5 (comprising the nucleotide sequence of SEQ ID NO. 37), marker M6 (comprising the nucleotide sequence of SEQ ID NO. 38) or marker M7 (comprising the nucleotide sequence of SEQ ID NO. 39) and a marker selected from marker M11 (comprising the nucleotide sequence of SEQ ID NO. 43), marker M12 (comprising the nucleotide sequence of SEQ ID NO. 44) or marker M13 (comprising the nucleotide sequence of SEQ ID NO. 45) or the small subunit of ALS may be encoded by a chromosomal region located on chromosome 4, between a marker selected from marker M8 (comprising the nucleotide sequence of SEQ ID NO. 40), marker M9 (comprising the nucleotide sequence of SEQ ID NO. 41) or marker M10 (comprising the nucleotide sequence of SEQ ID NO. 42) and a marker selected from marker M14 (comprising the nucleotide sequence of SEQ ID NO. 46), marker M15 (comprising the nucleotide sequence of SEQ ID NO. 47) or marker M16 (comprising the nucleotide sequence of SEQ ID NO. 48).

In another aspect, the invention provides an acetolactate synthase (ALS) inhibitor-herbicide tolerant Beta vulgaris plant, or hybrid seed comprising an ALS holo-enzyme comprising a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan, such as a large subunit of ALS comprising the amino acid sequence of SEQ ID NO. 3 or being encoded by a nucleotide sequence comprises the nucleotide sequence of SEQ ID NO. 4; and a small subunit of ALS comprising an amino acid sequence having at least 95%, 96%, 97%, 98% or 99% sequence identity with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31 or being encoded by a nucleotide sequence having at least 95%, 96%, 97%, 98% or 99% sequence identity with a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.

In yet another aspect, a method is provided for producing a Beta vulgaris plant with optimally fitted large subunit and one or more regulatory subunits of an ALS holoenzyme comprising the steps of a) crossing a Beta vulgaris plant comprising an allele encoding a large subunit of ALS comprising an amino acid sequence having at least 95%, 96%, 97%, 98% or 99% sequence identity with the amino acid sequence of SEQ ID NO. 1 and further comprising a leucine at a position corresponding to amino acid position 569 instead of the naturally occurring tryptophan, with a Beta vulgaris plant comprising at least one allele encoding a small subunit of ALS comprises an amino acid sequence having at least 95%, 96%, 97%, 98% or 99% sequence identity with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31; and identifying progeny plants comprising the allele encoding said large subunit of ALS and the at least one allele encoding said regulatory subunit of ALS.

In a further aspect, the invention provides a method for producing a Beta vulgaris plant with optimally fitted large subunit and one or more regulatory subunits of an ALS holoenzyme comprising the steps of providing a Beta vulgaris plant comprising an allele encoding a large subunit of ALS comprising an amino acid sequence having at least 95%, 96%, 97%, 98% or 99% sequence identity with the amino acid sequence of SEQ ID NO. 1 and further comprising a leucine at a position corresponding to amino acid position 569 instead of the naturally occurring tryptophan, such as an amino acid sequence of SEQ ID NO. 3; and adapting, by genome-editing or directed mutation, the nucleotide sequence of the allele on chromosome 3 and/or the allele on chromosome 4 encoding a small subunit of ALS to obtain a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.

In a further aspect the present invention provides a method for identifying or selecting an acetolactate synthase (ALS) inhibitor-herbicide tolerant Beta vulgaris plant, or hybrid seed, or a part thereof, with optimally fitted large subunit and one or more regulatory subunits of an ALS holoenzyme comprising the steps of identifying in a Beta vulgaris plant, or in a hybrid seed, or a part thereof a large subunit of ALS comprising an amino acid sequence having at least 95%, 96%, 97%, 98% or 99% sequence identity with the amino acid sequence of SEQ ID NO. 1 and further comprising a leucine at a position corresponding to amino acid position 569 instead of the naturally occurring tryptophan, such as a large subunit of ALS comprising the amino acid sequence of SEQ ID NO. 3 or being encoded by a nucleotide sequence comprises the nucleotide sequence of SEQ ID NO. 4; and identifying in said plant, said seed or said part a small subunit of ALS which can be selected by identification with marker M1 (comprising the nucleotide sequence of SEQ ID NO. 33), marker M2 (comprising the nucleotide sequence of SEQ ID NO. 34), marker M3 (comprising the nucleotide sequence of SEQ ID NO. 35) or marker M4 (comprising the nucleotide sequence of SEQ ID NO. 36), and optionally selecting the acetolactate synthase (ALS) inhibitor-herbicide tolerant Beta vulgaris plant, or hybrid seed, or a part thereof, with optimally fitted large subunit and one or more regulatory subunits of an ALS holoenzyme. The small subunit of ALS may be encoded by a chromosomal region located on chromosome 3, between a marker selected from marker M5 (comprising the nucleotide sequence of SEQ ID NO. 37), marker M6 (comprising the nucleotide sequence of SEQ ID NO. 38) or marker M7 (comprising the nucleotide sequence of SEQ ID NO. 39) and a marker selected from marker M11 (comprising the nucleotide sequence of SEQ ID NO. 43), marker M12 (comprising the nucleotide sequence of SEQ ID NO. 44) or marker M13 (comprising the nucleotide sequence of SEQ ID NO. 45) or the small subunit of ALS may be encoded by a chromosomal region located on chromosome 4, between a marker selected from marker M8 (comprising the nucleotide sequence of SEQ ID NO. 40), marker M9 (comprising the nucleotide sequence of SEQ ID NO. 41) or marker M10 (comprising the nucleotide sequence of SEQ ID NO. 42) and a marker selected from marker M14 (comprising the nucleotide sequence of SEQ ID NO. 46), marker M15 (comprising the nucleotide sequence of SEQ ID NO. 47) or marker M16 (comprising the nucleotide sequence of SEQ ID NO. 48).

The invention also provides use of Beta vulgaris plants as herein described for the production of sugar, ethanol, biogas, betaine and/or uridine or the use of Beta vulgaris plants to produce feed for animals or use of said Beta vulgaris plants as animal feed stuff.

It is another aspect of the invention to provide the use of one or more ALS inhibitor herbicide(s) for controlling unwanted vegetation in Beta vulgaris growing areas wherein the Beta vulgaris plants are hybrid Beta vulgaris plants as herein described. The ALS inhibitor herbicide(s) in combination with non-ALS inhibitor herbicides (i.e. herbicides showing a mode of action that is different to the inhibition of the ALS enzyme [acetohydroxyacid synthase; EC 2.2.1.6] group D herbicides), and wherein the non-ALS inhibitor herbicide(s) is/are selected form the group consisting of chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.

Further provided by the invention is a method for controlling unwanted vegetation in Beta vulgaris plant growing areas, characterized by (a) the presence of Beta vulgaris plants as herein described; (b) the application of one or more ALS inhibitor herbicide(s) alone or in combination with one or more herbicide(s) that do(es) not belong to the class of ALS inhibitor herbicides (non-ALS inhibitor herbicides), wherein the application of the respective herbicides as defined under (b) can (i) take place jointly or simultaneously, or (ii) take place at different times and/or in a plurality of portions (sequential application), in pre-emergence applications followed by post-emergence applications or early post-emergence applications followed by medium or late post-emergence applications.

Embodiments of the invention are reflected in the following numbered paragraphs:
Embodiment 1. An acetolactate synthase (ALS) inhibitor-herbicide Beta vulgaris plant comprising an ALS holo-enzyme comprising
   a. a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan; and
   b. a small subunit of ALS which can be selected by identification with marker M1 (comprising the nucleotide sequence of SEQ ID NO. 33), marker M2 (comprising the nucleotide sequence of SEQ ID NO. 34), marker M3 (comprising the nucleotide sequence of SEQ ID NO. 35) or marker M4 (comprising the nucleotide sequence of SEQ ID NO. 36).
Embodiment 2. The Beta vulgaris plant of embodiment 1, wherein said small subunit of ALS is encoded by a chromosomal region located on chromosome 3, between a marker selected from marker M5 (comprising the nucleotide sequence of SEQ ID NO. 37), marker M6 (comprising the nucleotide sequence of SEQ ID NO. 38) or marker M7 (comprising the nucleotide sequence of SEQ ID NO. 39) and a marker selected from marker M11 (comprising the nucleotide sequence of SEQ ID NO. 43), marker M12 (comprising the nucleotide sequence of SEQ ID NO. 44) or marker M13 (comprising the nucleotide sequence of SEQ ID NO. 45).
Embodiment 3. The Beta vulgaris plant of embodiment 1 or embodiment 2, wherein the small subunit of ALS is encoded by a chromosomal region located on chromosome 4, between a marker selected from marker M8 (comprising the nucleotide sequence of SEQ ID NO. 40), marker M9 (comprising the nucleotide sequence of SEQ ID NO. 41) or marker M10 (comprising the nucleotide sequence of SEQ ID NO. 42) and a marker selected from marker M14 (comprising the nucleotide sequence of SEQ ID NO. 46), marker M15 (comprising the nucleotide sequence of SEQ ID NO. 47) or marker M16 (comprising the nucleotide sequence of SEQ ID NO. 48).
Embodiment 4. The Beta vulgaris plant of any one of embodiments 1 to 3 wherein said small subunit of ALS comprises an amino acid sequence having at least 95% sequence identity with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31.
Embodiment 5. The Beta vulgaris plant of any one of embodiments 1 to 4, wherein the small subunit of ALS is encoded by a nucleotide sequence having at least 95% sequence identity with a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.
Embodiment 6. The Beta vulgaris plant of any one of embodiments 1 to 5 wherein said small subunit of ALS comprises an amino acid sequence having at least 98% sequence identity with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31.
Embodiment 7. The Beta vulgaris plant of any one of embodiments 1 to 6, wherein said small subunit of ALS is encoded by a nucleotide sequence having at least 98% sequence identity with a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.
Embodiment 8. The Beta vulgaris plant of any one of embodiments 1 to 7 wherein said small subunit of ALS comprises an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31.
Embodiment 9. The Beta vulgaris plant of any one of embodiments 1 to 8, wherein said small subunit of ALS is encoded by a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.
Embodiment 10. The Beta vulgaris plant of embodiment 1, comprising:
   a. an allele of a small subunit of ALS on chromosome 3 encoding an amino acid sequence having 98% sequence identity to the amino acid sequence of SEQ ID NO. 13 and further comprising an allele of a small subunit of ALS on chromosome 4 encoding an amino acid sequence having 98% sequence identity to the amino acid sequence of SEQ ID NO. 15;
   b. an allele of a small subunit of ALS on chromosome 3 encoding an amino acid sequence having 98% sequence identity to the amino acid sequence of SEQ ID NO. 17 and further comprising an allele of a small subunit of ALS on chromosome 4 encoding an amino acid sequence having 98% sequence identity to the amino acid sequence of SEQ ID NO. 19;
   c. an allele of a small subunit of ALS on chromosome 3 encoding an amino acid sequence having 98% sequence identity to the amino acid sequence of SEQ ID NO. 21 and further comprising an allele of a small subunit of ALS on chromosome 4 encoding an amino acid sequence having 98% sequence identity to the amino acid sequence of SEQ ID NO. 23;
   d. an allele of a small subunit of ALS on chromosome 3 encoding an amino acid sequence having 98% sequence identity to the amino acid sequence of SEQ ID NO. 25 and further comprising an allele of a small subunit of ALS on chromosome 4 encoding an amino acid sequence having 98% sequence identity to the amino acid sequence of SEQ ID NO. 27; or
   e. an allele of a small subunit of ALS on chromosome 3 encoding an amino acid sequence having 98% sequence identity to the amino acid sequence of SEQ ID NO. 29 and further comprising an allele of a small subunit of ALS on chromosome 4 encoding an amino acid sequence having 98% sequence identity to the amino acid sequence of SEQ ID NO. 31.
Embodiment 11. The Beta vulgaris plant of embodiment 1, comprising:
   a. an allele of a small subunit of ALS on chromosome 3 comprising a nucleotide sequence having 98% sequence identity to the nucleotide sequence of SEQ ID NO. 14 and further comprising an allele of a small subunit of ALS on chromosome 4 comprising a nucleotide sequence having 98% sequence identity to the nucleotide sequence of SEQ ID NO. 16;
   b. an allele of a small subunit of ALS on chromosome 3 comprising a nucleotide sequence having 98% sequence identity to the nucleotide sequence of SEQ ID NO. 18 and further comprising an allele of a small subunit of ALS on chromosome 4 comprising a nucleotide sequence having 98% sequence identity to the nucleotide sequence of SEQ ID NO. 20;
   c. an allele of a small subunit of ALS on chromosome 3 comprising a nucleotide sequence having 98% sequence identity to the nucleotide sequence of SEQ ID NO. 22 and further comprising an allele of a small subunit of ALS on chromosome 4 comprising a nucleotide sequence having 98% sequence identity to the nucleotide sequence of SEQ ID NO. 24;
   d. an allele of a small subunit of ALS on chromosome 3 comprising a nucleotide sequence having 98% sequence identity to the nucleotide sequence of SEQ ID NO. 26 and further comprising an allele of a small subunit of ALS on chromosome 4 comprising a nucleotide sequence having 98% sequence identity to the nucleotide sequence of SEQ ID NO. 28; or
   e. an allele of a small subunit of ALS on chromosome 3 comprising a nucleotide sequence having 98% sequence identity to the nucleotide sequence of SEQ ID NO. 30 and further comprising an allele of a small subunit of ALS on chromosome 4 comprising a nucleotide sequence having 98% sequence identity to the nucleotide sequence of SEQ ID NO. 32.
Embodiment 12. The Beta vulgaris plant of any one of embodiments 1 to 11, wherein said large subunit of ALS comprises the amino acid sequence of SEQ ID NO. 3.
Embodiment 13. The Beta vulgaris plant of any one of embodiments 1 to 12, wherein said large subunit of ALS is encoded by a nucleotide sequence comprises the nucleotide sequence of SEQ ID NO. 4.
Embodiment 14. The Beta vulgaris plant of any one of embodiments 1 to 13, wherein said large subunit of ALS is encoded by a homozygous nucleotide sequence.
Embodiment 15. The Beta vulgaris plant of any one of embodiments 1 to 14, wherein said small subunit of ALS is encoded by a homozygous nucleotide sequence.
Embodiment 16. The Beta vulgaris plant of any one of embodiments 1 to 15, which is a hybrid Beta vulgaris plant.
Embodiment 17. The Beta vulgaris plant or seed of any one of embodiments 1 to 16, which is sugar beet or sugar beet seed.
Embodiment 18. A method for producing a Beta vulgaris plant with optimally fitted large subunit and one or more regulatory subunits of an ALS holoenzyme comprising:
   a. crossing a Beta vulgaris plant comprising an allele encoding a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan, with a Beta vulgaris plant comprising at least one allele encoding a small subunit of ALS comprises an amino acid sequence having at least 95% sequence identity with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31; and
   b. identifying progeny plants comprising said allele encoding said large subunit of ALS and said at least one allele encoding said regulatory subunit of ALS.
Embodiment 19. The method of embodiment 18 wherein step b comprises identification of said at least one allele encoding said regulatory subunit of ALS using any one of marker M1 (comprising the nucleotide sequence of SEQ ID NO. 33), marker M2 (comprising the nucleotide sequence of SEQ ID NO. 34), marker M3 (comprising the nucleotide sequence of SEQ ID NO. 35), marker M4 (comprising the nucleotide sequence of SEQ ID NO. 36), marker M5 (comprising the nucleotide sequence of SEQ ID NO. 37), marker M6 (comprising the nucleotide sequence of SEQ ID NO. 38), marker M7 (comprising the nucleotide sequence of SEQ ID NO. 39), marker M1 (comprising the nucleotide sequence of SEQ ID NO. 43), marker M12 (comprising the nucleotide sequence of SEQ ID NO. 44), marker M13 (comprising the nucleotide sequence of SEQ ID NO. 45), marker M8 (comprising the nucleotide sequence of SEQ ID NO. 40), marker M9 (comprising the nucleotide sequence of SEQ ID NO. 41), marker M10 (comprising the nucleotide sequence of SEQ ID NO. 42), marker M14 (comprising the nucleotide sequence of SEQ ID NO. 46), marker M15 (comprising the nucleotide sequence of SEQ ID NO. 47) or marker M16 (comprising the nucleotide sequence of SEQ ID NO. 48) Embodiment 20. A method for producing a Beta vulgaris plant with optimally fitted large subunit and one or more regulatory subunits of an ALS holoenzyme comprising:
   c. providing a Beta vulgaris plant comprising an allele encoding a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan, such as an amino acid sequence of SEQ ID NO. 3;
   d. adapting, by genome-editing or directed mutation, the nucleotide sequence of the allele on chromosome 3 and/or the allele on chromosome 4 encoding a small subunit of ALS to obtain a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.
Embodiment 21. Use of a Beta vulgaris plant according to any one of embodiments 1 to 17 for the production of sugar, ethanol, betaine and/or uridine or for the production of animal feed.
Embodiment 22. Use of one or more ALS inhibitor herbicide(s) for controlling unwanted vegetation in Beta vulgaris growing areas wherein the Beta vulgaris plants are Beta vulgaris plants according to embodiments 1 to 17.
Embodiment 23. Use of one or more ALS inhibitor herbicide(s) according to embodiment 22, wherein the ALS inhibitor herbicide(s) belong(s) to:
   the group of the (sulfon)amides (group (A)) consisting of:
      the subgroup (A1) of the sulfonylureas, consisting of:
      amidosulfuron [CAS RN 120923-37-7] (=A1-1);
      azimsulfuron [CAS RN 120162-55-2] (=A1-2);
      bensulfuron-methyl [CAS RN 83055-99-6] (=A1-3);
      chlorimuron-ethyl [CAS RN 90982-32-4] (=A1-4);
      chlorsulfuron [CAS RN 64902-72-3] (=A1-5);
      cinosulfuron [CAS RN 94593-91-6] (=A1-6);
      cyclosulfamuron [CAS RN 136849-15-5] (=A1-7);
      ethametsulfuron-methyl [CAS RN 97780-06-8] (=A1-8);
      ethoxysulfuron [CAS RN 126801-58-9] (=A1-9);
      flazasulfuron [CAS RN 104040-78-0] (=A1-10);
      flucetosulfuron [CAS RN 412928-75-7] (=A1-11);
      flupyrsulfuron-methyl-sodium [CAS RN 144740-54-5] (=A1-12);
      foramsulfuron [CAS RN 173159-57-4] (=A1-13);
      halosulfuron-methyl [CAS RN 100784-20-1] (=A1-14);
      imazosulfuron [CAS RN 122548-33-8] (=A1-15);
      iodosulfuron-methyl-sodium [CAS RN 144550-36-7] (=A1-16);
      mesosulfuron-methyl [CAS RN 208465-21-8] (=A1-17);
      metsulfuron-methyl [CAS RN 74223-64-6] (=A1-18);
      monosulfuron [CAS RN 155860-63-2] (=A1-19);
      nicosulfuron [CAS RN 111991-09-4] (=A1-20);
      orthosulfamuron [CAS RN 213464-77-8] (=A1-21);
      oxasulfuron [CAS RN 144651-06-9] (=A1-22);
      primisulfuron-methyl [CAS RN 86209-51-0] (=A1-23);
      prosulfuron [CAS RN 94125-34-5] (=A1-24);
      pyrazosulfuron-ethyl [CAS RN 93697-74-6] (=A1-25);
      rimsulfuron [CAS RN 122931-48-0] (=A1-26);
      sulfometuron-methyl [CAS RN 74222-97-2] (=A1-27);
      sulfosulfuron [CAS RN 141776-32-1] (=A1-28);
      thifensulfuron-methyl [CAS RN 79277-27-3] (=A1-29);
      triasulfuron [CAS RN 82097-50-5] (=A1-30);
      tribenuron-methyl [CAS RN 101200-48-0] (=A1-31);
      trifloxysulfuron [CAS RN 145099-21-4] (sodium) (=A1-32);
      triflusulfuron-methyl [CAS RN 126535-15-7] (=A1-33);
      tritosulfuron [CAS RN 142469-14-5] (=A1-34);
      NC-330 [CAS RN 104770-29-8] (=A1-35);
      NC-620 [CAS RN 868680-84-6] (=A1-36);
      TH-547 [CAS RN 570415-88-2] (=A1-37);
      monosulfuron-methyl [CAS RN 175076-90-1] (=A1-38);
      2-iodo-N-[(4-methoxy-6-methyl-1,3,5-triazinyl)carbamoyl]benzene-sulfonamide (=A1-39);
      a compound of the general formula (I)

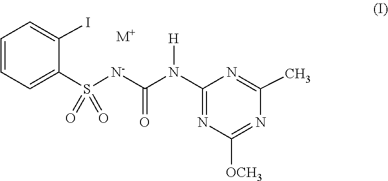

where M+ denotes the respective salt of the compound (I), i.e. its lithium salt (=A1-40); its sodium salt (=A1-41); its potassium salt (=A1-42); its magnesium salt (=A1-43); its calcium (=A1-44); its ammonium salt (=A1-45); its methylammonium salt (=A1-46); its dimethylammonium salt (=A1-47); its tetramethylammonium salt (=A1-48); its ethylammonium salt (=A1-49); its diethylammonium salt (=A1-50); its tetraethylammonium salt (=A1-51); its propylammonium salt (=A1-52); its tetrapropylammonium salt (=A1-53); its isopropylammonium salt (=A1-54); its diisopropylammonium salt (=A1-55); its butylammonium salt (=A1-56); its tetrabutylammonium salt (=A1-57); its (2-hydroxyeth-1-yl)ammonium salt (=A1-58); its bis-N,N-(2-hydroxyeth-1-yl)ammonium salt (=A1-59); its tris-N,N,N-(2-hydroxyeth-1-yl)ammonium salt (=A1-60); its 1-phenylethylammonium salt (=A1-61); its 2-phenylethylammonium salt (=A1-62); its trimethylsulfonium salt (=A1-63); its trimethyloxonium salt (=A1-64); its pyridinium salt (=A1-65); its 2-methylpyridinium salt (=A1-66); its 4-methylpyridinium salt (=A1-67); its 2,4-dimethylpyridinium salt (=A1-68); its 2,6-dimethylpyridinium salt (=A1-69); its piperidinium salt (=A1-70); its imidazolium salt (=A1-71); its morpholinium salt (=A1-72); its 1,5-diazabicyclo[4.3.0]non-7-enium salt (=A1-73); its 1,8-diazabicyclo[5.4.0]undec-7-enium salt (=A1-74);
or a compound of the formula (II) or salts thereof

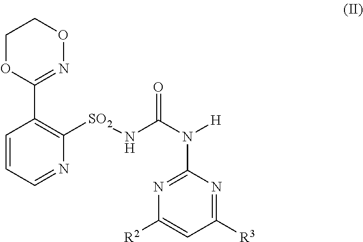

with R2, and R3 having the meaning as defined in the below table

| Compound | R² | R³ |
|---|---|---|
| A1-75 | OCH₃ | OC₂H₅ |
| A1-76 | OCH₃ | CH₃ |
| A1-77 | OCH₃ | C₂H₅ |
| A1-78 | OCH₃ | CF₃ |
| A1-79 | OCH₃ | OCF₂H |
| A1-80 | OCH₃ | NHCH₃ |
| A1-81 | OCH₃ | N(CH₃)₂ |
| A1-82 | OCH₃ | Cl |
| A1-83 | OCH₃ | OCH₃ |
| A1-84 | OC₂H₅ | OC₂H₅ |
| A1-85 | OC₂H₅ | CH₃ |
| A1-86 | OC₂H₅ | C₂H₅ |

or the compound of formula (III) (=A1-87), i.e. the sodium salt of compound (A1-83)

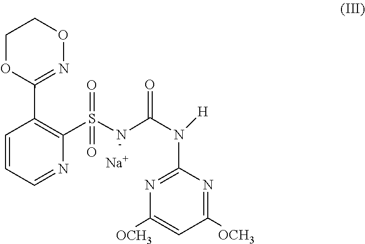 [SMILES: COc1cc(OC)nc(NC(=O)NS(=O)(=O)c2ncccc2C2=NOCCO2)n1 ; [Na+]]

or the compound of formula (IV) (=A1-88), i.e. the sodium salt of compound (A1-82)

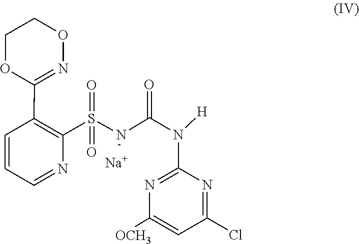 [SMILES: COc1cc(Cl)nc(NC(=O)NS(=O)(=O)c2ncccc2C2=NOCCO2)n1 ; [Na+]]

the subgroup of the sulfonylaminocarbonyltriazolinones (subgroup ((A2)), consisting of:
   flucarbazone-sodium [CAS RN 181274-17-9] (=A2-1);
   propoxycarbazone-sodium [CAS RN 181274-15-7] (=A2-2);
   thiencarbazone-methyl [CAS RN 317815-83-1] (=A2-3);
the subgroup of the triazolopyrimidines (subgroup (A3)), consisting of:
   cloransulam-methyl [147150-35-4] (=A3-1);
   diclosulam [CAS RN 145701-21-9] (=A3-2);
   florasulam [CAS RN 145701-23-1] (=A3-3);
   flumetsulam [CAS RN 98967-40-9] (=A3-4);
   metosulam [CAS RN 139528-85-1] (=A3-5);
   penoxsulam [CAS RN 219714-96-2] (=A3-6);
   pyroxsulam [CAS RN 422556-08-9] (=A3-7);
the subgroup of the sulfonanilides (subgroup (A4)), consisting of:
   compounds or salts thereof from the group described by the general formula (V):

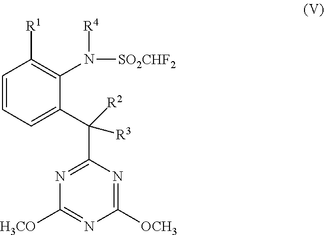

in which
R1 is halogen, preferably fluorine or chlorine,
R2 is hydrogen and R3 is hydroxyl or
R2 and R3 together with the carbon atom to which they are attached are a carbonyl group C═O and
R4 is hydrogen or methyl;
and more especially compounds of the below given chemical structure (A4-1) to (A4-8)

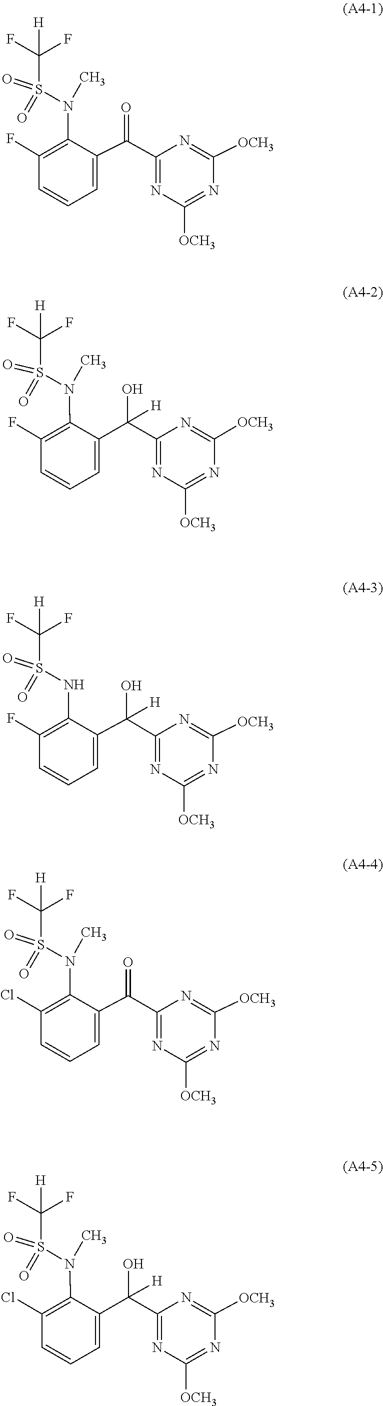 [SMILES: COc1nc(OC)nc(C(=O)c2cccc(F)c2N(C)S(=O)(=O)C(F)F)n1 ; COc1nc(OC)nc(C(O)c2cccc(F)c2N(C)S(=O)(=O)C(F)F)n1 ; COc1nc(OC)nc(C(O)c2cccc(F)c2NS(=O)(=O)C(F)F)n1 ; COc1nc(OC)nc(C(=O)c2cccc(Cl)c2N(C)S(=O)(=O)C(F)F)n1 ; COc1nc(OC)nc(C(O)c2cccc(Cl)c2N(C)S(=O)(=O)C(F)F)n1]

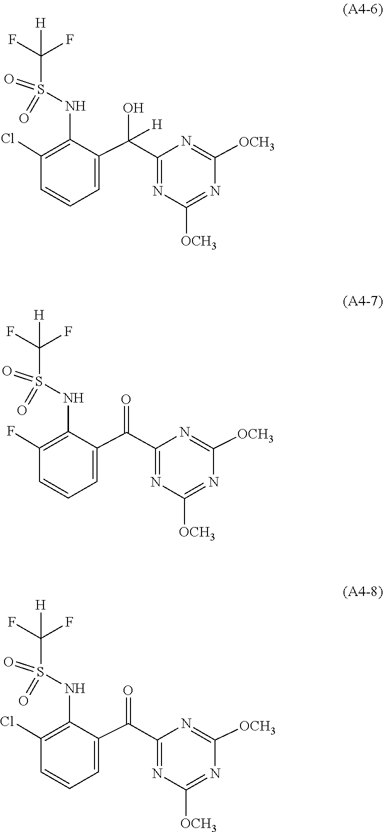 [SMILES: COc1nc(OC)nc(C(O)c2cccc(Cl)c2NS(=O)(=O)C(F)F)n1 ; COc1nc(OC)nc(C(=O)c2cccc(F)c2NS(=O)(=O)C(F)F)n1 ; COc1nc(OC)nc(C(=O)c2cccc(Cl)c2NS(=O)(=O)C(F)F)n1]

the group of the imidazolinones (group (B1)), consisting of:
   imazamethabenzmethyl [CAS RN 81405-85-8] (=B1-1);
   imazamox [CAS RN 114311-32-9] (=B1-2);
   imazapic [CAS RN 104098-48-8] (=B1-3);
   imazapyr [CAS RN 81334-34-1] (=B1-4);
   imazaquin [CAS RN 81335-37-7] (=B1-5);
   imazethapyr [CAS RN 81335-77-5] (=B1-6);
   SYP-298 [CAS RN 557064-77-4] (=B1-7);
   SYP-300 [CAS RN 374718-10-2] (=B1-8);
the group of the pyrimidinyl(thio)benzoates (group (C)), consisting of:
   the subgroup of the pyrimidinyloxybenzoeacids (subgroup (C1)) consisting of:
      bispyribac-sodium [CAS RN 125401-92-5] (=C1-1);
      pyribenzoxim [CAS RN 168088-61-7] (=C1-2);
      pyriminobac-methyl [CAS RN 136191-64-5] (=C1-3);
      pyribambenz-isopropyl [CAS RN 420138-41-6] (=C1-4);
      pyribambenz-propyl [CAS RN 420138-40-5] (=C1-5);
   the subgroup of the pyrimidinylthiobenzoeacids (subgroup (C2)), consisting of:
      pyriftalid [CAS RN 135186-78-6] (=C2-1);
      pyrithiobac-sodium [CAS RN 123343-16-8] (=C2-2).
Embodiment 24. Use of one or more ALS inhibitor herbicide(s) according to embodiment 22 or 23, wherein the ALS inhibitor herbicide(s) belong(s) to the group consisting of:
   amidosulfuron [CAS RN 120923-37-7] (=A1-1);
   chlorimuron-ethyl [CAS RN 90982-32-4] (=A1-4);
   ethametsulfuron-methyl [CAS RN 97780-06-8] (=A1-8);
   ethoxysulfuron [CAS RN 126801-58-9] (=A1-9);
   flupyrsulfuron-methyl-sodium [CAS RN 144740-54-5] (=A1-12);
   foramsulfuron [CAS RN 173159-57-4] (=A1-13);
   iodosulfuron-methyl-sodium [CAS RN 144550-36-7] (=A1-16);
   mesosulfuron-methyl [CAS RN 208465-21-8] (=A1-17);
   metsulfuron-methyl [CAS RN 74223-64-6] (=A1-18);
   monosulfuron [CAS RN 155860-63-2] (=A1-19);
   nicosulfuron [CAS RN 111991-09-4] (=A1-20);
   sulfosulfuron [CAS RN 141776-32-1] (=A1-28);
   thifensulfuron-methyl [CAS RN 79277-27-3] (=A1-29);
   tribenuron-methyl [CAS RN 101200-48-0] (=A1-31);
   2-iodo-N-[(4-methoxy-6-methyl-1,3,5-triazinyl)carbamoyl]benzene-sulfonamide (=A1-39);
   2-iodo-N-[(4-methoxy-6-methyl-1,3,5-triazinyl)carbamoyl]benzene-sulfonamide sodium salt (=A1-41);
   (A1-83) or its sodium salt (=A1-87);
   propoxycarbazone-sodium [CAS RN 181274-15-7] (=A2-2);
   thiencarbazone-methyl [CAS RN 317815-83-1] (=A2-3);
   florasulam [CAS RN 145701-23-1] (=A3-3);
   metosulam [CAS RN 139528-85-1] (=A3-5);
   pyroxsulam [CAS RN 422556-08-9] (=A3-7) (A4-1);
   (A4-2);
   (A4-3);
   imazamox [CAS RN 114311-32-9] (=B1-2); and
   bispyribac-sodium [CAS RN 125401-92-5] (=C1-1).
Embodiment 25. Use of one or more ALS inhibitor herbicide(s) according to embodiment 22 or 23, wherein the ALS inhibitor herbicide(s) belong(s) to the group consisting of:
   amidosulfuron [CAS RN 120923-37-7] (=A1-1);
   foramsulfuron [CAS RN 173159-57-4] (=A1-13);
   iodosulfuron-methyl-sodium [CAS RN 144550-36-7] (=A1-16);
   2-iodo-N-[(4-methoxy-6-methyl-1,3,5-triazinyl)carbamoyl]benzene-sulfonamide (=A1-39);
   2-iodo-N-[(4-methoxy-6-methyl-1,3,5-triazinyl)carbamoyl]benzene-sulfonamide sodium salt (=A1-41);
   A1-83 or its sodium salt (=A1-87);
   thiencarbazone-methyl [CAS RN 317815-83-1] (=A2-3);
   imazamox [CAS RN 114311-32-9] (=B1-2);
   bispyribac-sodium [CAS RN 125401-92-5] (=C1-1).
Embodiment 26. Use of one or more ALS inhibitor herbicide(s) according to embodiment 22 or 23, wherein the ALS inhibitor herbicide(s) comprises foramsulfuron [CAS RN 173159-57-4] (=A1-13) and thiencarbazone-methyl [CAS RN 317815-83-1] (=A2-3).
Embodiment 27. Use of one or more ALS inhibitor herbicide(s) according to any of embodiments 22 to 26 in combination with non-ALS inhibitor herbicides (i.e. herbicides showing a mode of action that is different to the inhibition of the ALS enzyme [acetohydroxyacid synthase; EC 2.2.1.6] group D herbicides), and wherein the non-ALS inhibitor herbicide(s) is/are selected form the group consisting of:
   chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.
Embodiment 28. Use of one or more ALS inhibitor herbicide(s) according to embodiment 27, wherein the non-ALS inhibitor herbicide(s) is/are selected form the group consisting of:
   desmedipham, ethofumesate, glufosinate, glufosinate-ammonium, glufosinate-P, glufosinate-P-ammonium, glufosinate-P-sodium, glyphosate, glyphosate-isopropylammonium, lenacil, metamitron, phenmedipham, phenmedipham-ethyl.
Embodiment 29. Method for controlling unwanted vegetation in Beta vulgaris plant growing areas, characterized by:
   (a) the presence of Beta vulgaris plants according to any one of embodiments 1 to 17
(b) the application of one or more ALS inhibitor herbicide(s) alone or in combination with one or more herbicide(s) that do(es) not belong to the class of ALS inhibitor herbicides (non-ALS inhibitor herbicides), and
(c) wherein the application of the respective herbicides as defined under (b)
   (i) takes place jointly or simultaneously, or
   (ii) takes place at different times and/or in a plurality of portions (sequential application), in pre-emergence applications followed by post-emergence applications or early post-emergence applications followed by medium or late post-emergence applications.
Embodiment 30. Method according to claim 29 for controlling unwanted vegetation, wherein the ALS inhibitor herbicide(s) are taken from the groups as defined in embodiment 23.
Embodiment 31. Method according to claim 28, wherein the ALS inhibitor herbicide(s) are taken from the groups as defined in embodiment 24.
Embodiment 32. Method according to embodiment 29 for controlling unwanted vegetation, and wherein the ALS inhibitor herbicide(s) comprise foramsulfuron [CAS RN 173159-57-4] (=A1-13) and thiencarbazone-methyl [CAS RN 317815-83-1] (=A2-3).
Embodiment 33. Method according to any one of embodiments 29 to 32, wherein the non-ALS inhibitor herbicide(s) are taken from the group consisting of:
   chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.

### DETAILED DESCRIPTION OF THE EMBODIMENTS

The inventors have unexpectedly discovered natural variation in the beet regulatory ALS subunits and identified the best fitting combinations of regulatory subunits and catalytical subunits possessing herbicide-tolerant mutations to gain robust BvALS enzyme performance.

More in particular, the inventors have identified the orthologous genes in Beta vulgaris corresponding to genes coding for the ALS regulatory subunits in Arabidopsis thaliana At2g31810 (amino acid sequence SEQ ID NO. 5; nucleotide sequence SEQ ID NO. 6) and At5g16290 (amino acid sequence SEQ ID NO.7; nucleotide sequence SEQ ID NO. 8). The Beta vulgaris gene corresponding to At2g31810 is located on chromosome 3 and is hereinafter referred to as BV3_059040, while the Beta vulgaris gene corresponding to At5g16290 is located on chromosome 4 and is hereinafter referred to as BV4_074570. The amino acid sequence of the regulatory subunit encoded by BV3_05904, as can be found in the reference Beta vulgaris genome, is represented in the sequence listing SEQ ID NO. 9 and the nucleotide sequence in SEQ ID NO. 10. The amino acid sequence of the regulatory subunit encoded by BV4_074570, as can be found in the reference Beta vulgaris genome, is represented in the sequence listing SEQ ID NO. 11 and the nucleotide sequence in SEQ ID NO. 12.

Sequence listing entries SEQ ID NO. 13, SEQ ID NO. 17, SEQ ID NO 21, SEQ ID NO. 25 and SEQ ID NO. 29 represent amino acid sequences for variant alleles of BV3_05904 as can be found in B. vulgaris genotype A, B. vulgaris genotype B, B. vulgaris genotype C, B. vulgaris genotype D and B. vulgaris genotype E respectively.

Sequence listing entries SEQ ID NO. 15, SEQ ID NO. 19, SEQ ID NO. 23, SEQ ID NO. 27 and SEQ ID NO. 31 represent amino acid sequences for variant alleles of BV4_074570 as can be found in B. vulgaris genotype A; B. vulgaris genotype B; C. vulgaris genotype C, B. vulgaris genotype D and B. vulgaris genotype E respectively.

Sequence listing entries SEQ ID NO. 14, SEQ ID NO. 18, SEQ ID NO 22, SEQ ID NO. 26 and SEQ ID NO. 30 represent nucleotide sequences for variant alleles of BV3_05904 as can be found in B. vulgaris genotype A, B. vulgaris genotype B, B. vulgaris genotype C, B. vulgaris genotype D and B. vulgaris genotype E respectively.

Sequence listing entries SEQ ID NO. 16, SEQ ID NO. 20, SEQ ID NO. 24, SEQ ID NO. 28 and SEQ ID NO. 32 represent nucleotide sequences for variant alleles of BV4_074570 as can be found in B. vulgaris genotype A, B. vulgaris genotype B, B. vulgaris genotype C, B. vulgaris genotype D and B. vulgaris genotype E respectively.

Thus in a first aspect, the invention provides an ALS inhibitor herbicide tolerant Beta vulgaris plant or seed comprising an ALS holo-enzyme comprising a large subunit of ALS comprising an amino acid sequence having at least 95% or at least 96% or at least 97% or at least 98% or at least 99% sequence identity with, or is identical with, the amino acid sequence of SEQ ID NO. 1 and further comprising a leucine at a position corresponding to amino acid position 569 instead of the naturally occurring tryptophan; and a small subunit of ALS comprising an amino acid sequence having at least 95% or at least 96% or at least 97% or at least 98% or at least 99% sequence identity with, or is identical with, with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31. The small subunit may be encoded by a nucleotide sequence having at least 95% or at least 96% or at least 97% or at least 98% or at least 99% sequence identity with, or is identical with, with a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.

The Beta vulgaris plants described herein may comprise both Bv3_059040 and Bv4_074570 from the same genotypes A to E. The Beta vulgaris plants may comprise in addition to the herbicide tolerant large subunit of ALS, an allele of a small subunit of ALS on chromosome 3 encoding an amino acid sequence having 98% sequence identity to the amino acid sequence of SEQ ID NO. 13 and further comprising an allele of a small subunit of ALS on chromosome 4 encoding an amino acid sequence having 98% sequence identity to the amino acid sequence of SEQ ID NO. 15; or an allele of a small subunit of ALS on chromosome 3 encoding an amino acid sequence having 98% sequence identity to the amino acid sequence of SEQ ID NO. 17 and further comprising an allele of a small subunit of ALS on chromosome 4 encoding an amino acid sequence having 98% sequence identity to the amino acid sequence of SEQ ID NO. 19; or an allele of a small subunit of ALS on chromosome 3 encoding an amino acid sequence having 98% sequence identity to the amino acid sequence of SEQ ID NO. 21 and further comprising an allele of a small subunit of ALS on chromosome 4 encoding an amino acid sequence having 98% sequence identity to the amino acid sequence of SEQ ID NO. 23; or an allele of a small subunit of ALS on chromosome 3 encoding an amino acid sequence having 98% sequence identity to the amino acid sequence of SEQ ID NO. 25 and further comprising an allele of a small subunit of ALS on chromosome 4 encoding an amino acid sequence having 98% sequence identity to the amino acid sequence of SEQ ID NO. 27; or an allele of a small subunit of ALS on chromosome 3 encoding an amino acid sequence having 98% sequence identity to the amino acid sequence of SEQ ID NO. 29 and further comprising an allele of a small subunit of ALS on chromosome 4 encoding an amino acid sequence having 98% sequence identity to the amino acid sequence of SEQ ID NO. 31.

A preferred herbicide tolerant large subunit of the ALS holoenzyme is encoded by allele BvALS_W569L. As used herein, a BvALS_W569L allele is a mutant allele of an endogenous Beta vulgaris ALS gene, encoding an ALS protein wherein the amino acid at position 569 is leucine instead of the normally occurring tryptophan. Such a mutant allele endows Beta vulgaris plants comprising it with tolerance to various ALS inhibitor herbicides, as hereinafter described in more detail. An ALS protein wherein the amino acid at position 569 is leucine instead of the normally occurring tryptophan is set forth in SEQ ID NO 3. The ALS protein wherein the amino acid at position 569 is leucine instead of the normally occurring tryptophan may however vary at other amino acid positions than position 569 and may have an amino acid sequence which is has at least 90, 95, 97, 98, or 99% sequence identity or is 100% identical to the polypeptide or protein encoded by BvALS_W697L as set forth in SEQ ID NO 3 provided it contains a leucine at position 569 of the amino acid sequence.

The BvALS_W569L allele may comprise the nucleotide sequence of SEQ ID NO: 4 wherein a transversion of the “G” nucleotide at a position corresponding to position 1706 to a “T” nucleotide occurred compared to the wild type allele. The ALS allele may also vary at other nucleotide position than position 1706 and may have a nucleotide sequence which is at least 90, 95, 97, 98, or 99% sequence identity or is 100% identical to the nucleotide sequence of BvALS_W697L as set forth in SEQ ID NO 4 provided it contains a TTG codon at position 1705-1707 of SEQ ID NO 4.

Beta vulgaris plants comprising the BvALS_W569L allele are less sensitive to an ALS inhibitor, more preferably it is at least 100 times less sensitive, more preferably, 500 times, even more preferably 1000 times and most preferably less than 2000 times than Beta vulgaris plants comprising the wild-type allele. Less sensitive when used herein may, vice versa, be seen as “more tolerable” or “more resistant”. Similarly, “more tolerable” or “more resistant” may, vice versa, be seen as “less sensitive”. For example, B. vulgaris plants comprising the BvALS_W569L allele are at least 2000 times less sensitive to the ALS inhibitor herbicide foramsulfuron (a member of the ALS inhibitor subclass “sulfonylurea herbicides”) compared to B. vulgaris plants comprising a BvALS wild type allele.

As used herein a BvALS-WT or “wild-type allele,” “wild-type ALS allele”, “wild-type ALS gene” or “wild-type ALS polynucleotide” refer to a nucleotide sequence that encodes an ALS protein that lacks the W569L substitution. Reference nucleotide sequences and amino acid sequences corresponding to such BvALS_WT or encoded protein are set forth in SEQ ID NO 2 and SEQ ID NO: 1 respectively.

Preferably, the BvALS_W569L comprise only the substitution at position 569 of the encoded ALS protein as sole mutation. Reference B. vulgaris seeds comprising the BvALS_W569L allele have been deposited as NCIMB 41705.

Genes encoding small subunits of ALS can be identified with marker M1 (comprising the nucleotide sequence of SEQ ID NO. 33), marker M2 (comprising the nucleotide sequence of SEQ ID NO. 34) and marker M3 (comprising the nucleotide sequence of SEQ ID NO. 35) for Bv3_059040 or marker M4 (comprising the nucleotide sequence of SEQ ID NO. 36) for Bv4_074570.

The small subunit of ALS Bv3_059040 is encoded by a chromosomal region located on chromosome 3, between a marker selected from marker M5 (comprising the nucleotide sequence of SEQ ID NO. 37), marker M6 (comprising the nucleotide sequence of SEQ ID NO. 38) or marker M7 (comprising the nucleotide sequence of SEQ ID NO. 39) and a marker selected from marker M11 (comprising the nucleotide sequence of SEQ ID NO. 43), marker M12 (comprising the nucleotide sequence of SEQ ID NO. 44) or marker M13 (comprising the nucleotide sequence of SEQ ID NO. 45).

The small subunit of ALS Bv4_074570 is encoded by a chromosomal region located on chromosome 4, between a marker selected from marker M8 (comprising the nucleotide sequence of SEQ ID NO. 40), marker M9 (comprising the nucleotide sequence of SEQ ID NO. 41) or marker M10 (comprising the nucleotide sequence of SEQ ID NO. 42) and a marker selected from marker M14 (comprising the nucleotide sequence of SEQ ID NO. 46), marker M15 (comprising the nucleotide sequence of SEQ ID NO. 47) or marker M16 (comprising the nucleotide sequence of SEQ ID NO. 48).

Thus, in another aspect of the invention, a Beta vulgaris plant or seed is provided comprising an ALS holo-enzyme comprising a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan; and a small subunit of ALS which can be selected by identification with marker M1 (comprising the nucleotide sequence of SEQ ID NO. 33), marker M2 (comprising the nucleotide sequence of SEQ ID NO. 34), marker M3 (comprising the nucleotide sequence of SEQ ID NO. 35) or marker M4 (comprising the nucleotide sequence of SEQ ID NO. 36), particularly wherein said small subunit of ALS is encoded by a chromosomal region located on chromosome 3, between a marker selected from marker M5 (comprising the nucleotide sequence of SEQ ID NO. 37), marker M6 (comprising the nucleotide sequence of SEQ ID NO. 38) or marker M7 (comprising the nucleotide sequence of SEQ ID NO. 39) and a marker selected from marker M11 (comprising the nucleotide sequence of SEQ ID NO. 43), marker M12 (comprising the nucleotide sequence of SEQ ID NO. 44) or marker M13 (comprising the nucleotide sequence of SEQ ID NO. 45) and/or wherein the small subunit of ALS is encoded by a chromosomal region located on chromosome 4, between a marker selected from marker M8 (comprising the nucleotide sequence of SEQ ID NO. 40), marker M9 (comprising the nucleotide sequence of SEQ ID NO. 41) or marker M10 (comprising the nucleotide sequence of SEQ ID NO. 42) and a marker selected from marker M14 (comprising the nucleotide sequence of SEQ ID NO. 46), marker M15 (comprising the nucleotide sequence of SEQ ID NO. 47) or marker M16 (comprising the nucleotide sequence of SEQ ID NO. 48).

A genomic sequence build of the complete sequence for beets is publicly available and can be found at EnsemblPlants website https://plants.ensembl.org/Beta_vulgaris/Info/Index as RefBeet 1.2.2 Accession GCA_000511025 (https://www.ebi.ac.uk/ena/browser/view/GCA_000511025.2). A person skilled in the art can thus easily locate the marker nucleotide sequences herein provided on the complete genome sequence of beets, by sequence comparison using computer programs and algorithms. For example, BLAST, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be used to search for local sequence alignments.

Once the nucleotide sequence of the markers herein described have been physically allocated to the corresponding position in the genomic nucleotide sequence map, the person skilled in the art can identify the physical size of the chromosome 3 or 4 fragments flanked by such markers in kilobases, and can deduce the consensus nucleotide sequence of the fragment flanked by such markers.

Markers can be used to identify plants of the present invention using any genotypic analysis method. Genotypic evaluation of the plants includes using techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Allele-specific PCR (AS-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as “Microsatellites”. Additional compositions and methods for analyzing the genotype of the plants provided herein include those methods disclosed in U.S. Publication No. 2004/0171027, U.S. Publication No. 2005/02080506, and U.S. Publication No. 2005/0283858.

A particular useful assay method for genotyping of single nucleotide polymorphism markers is the KASP assay (Competitive allele specific PCR) as described e.g. by Chunlin He, John Holme and Jeffrey Anthony in “SNP genotyping: the KASP assay” Methods Mol Biol 2014; 1145:75-86 doi: 10.1007/978-1-4939-0446-4_7.

In the Beta vulgaris plants, and seed provided herein, the allele encoding the catalytic subunit or the allele(s) encoding regulatory subunits may be present in homozygous or heterozygous state. As used herein, “homozygous” or “homozygously” indicates that a plant has a copy of the same allele at the same locus on each of the corresponding chromosomes of the diploid pair of chromosomes. As used herein, “heterozygous” or “heterozygously” indicates that a plant has a copy of the different allele at the same locus on each of the corresponding chromosomes of the diploid pair of chromosomes.

Also described are Beta vulgaris plants, particularly elite Beta vulgaris plants, such as sugar-beet plants which comprise the alleles or DNA molecules as herein described, in homozygous or heterozygous state, and which can be used as parent plant to obtain the hybrid Beta vulgaris plants or seeds described herein. To this end, such plants are cross-pollinated and the progeny seeds are harvested. One of the parent plants may be male-sterile (female plant) and be pollinated with pollen from the male parent plant. Methods to obtain male-sterile Beta vulgaris plants are well known in the art.

The B. vulgaris plants of the present invention and harvestable parts thereof are agronomically exploitable. “Agronomically exploitable” means that the B. vulgaris plants and parts thereof are useful for agronomical purposes. For example, the B. vulgaris plants should serve for the purpose of being useful for sugar production, bio fuel production (such as biogas, biobutanol), ethanol production, betaine and/or uridine production and use of a Beta vulgaris plant as herein described for the production of sugar, ethanol, betaine and/or uridine is also envisioned. The B. vulgaris plants or parts thereof could be used to produce animal feed.

A “plant of the species Beta vulgaris” or a “Beta vulgaris plant” is, in particular, a plant of the subspecies Beta vulgaris subsp. vulgaris. For example, numbering among these are Beta vulgaris subsp. vulgaris var. altissima (sugar beet in a narrower sense), Beta vulgaris ssp. vulgaris var. vulgaris (chard), Beta vulgaris ssp. vulgaris var. conditiva (beetroot/red beet), Beta vulgaris ssp. vulgaris var. crassa/alba (fodder beet).

An example for an agronomically exploitable B. vulgaris plant is sugar beet. A sugar beet plant of the present invention when cultivated in an area of one hectare yields (about 80,000 to 90,000 sugar beets) should preferably serve for the production of at least 4 tons of sugar.

A sugar beet plant of the present invention should preferably contain a sugar content between 15-20%, preferably at least 17% so as to be agronomically exploitable. Thus, sugar beet plants that contain a sugar content between 15-20%, preferably at least 17% are a preferred embodiment of the present invention.

Yet another example of an agronomically exploitable B. vulgaris plant is fodder beet.

Another aspect of the present invention is the use of the Beta vulgaris plants described herein and/or the harvestable parts or propagation material described herein for the manufacture/breeding of further Beta vulgaris plants.

In another aspect of the present invention, a method is provided for producing a Beta vulgaris plant with optimally fitted large subunit and one or more regulatory subunits of an ALS holoenzyme comprising the steps of a) crossing a Beta vulgaris plant comprising an allele encoding a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan, with a Beta vulgaris plant comprising at least one allele encoding a small subunit of ALS comprises an amino acid sequence having at least 95% sequence identity with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31; and identifying progeny plants comprising said allele encoding said large subunit of ALS and said at least one allele encoding said regulatory subunit of ALS.

The identification of said at least one allele encoding said regulatory subunit of ALS can be achieved using any one of marker M1 (comprising the nucleotide sequence of SEQ ID NO. 33), marker M2 (comprising the nucleotide sequence of SEQ ID NO. 34), marker M3 (comprising the nucleotide sequence of SEQ ID NO. 35), marker M4 (comprising the nucleotide sequence of SEQ ID NO. 36), marker M5 (comprising the nucleotide sequence of SEQ ID NO. 37), marker M6 (comprising the nucleotide sequence of SEQ ID NO. 38), marker M7 (comprising the nucleotide sequence of SEQ ID NO. 39), marker M11 (comprising the nucleotide sequence of SEQ ID NO. 43), marker M12 (comprising the nucleotide sequence of SEQ ID NO. 44), marker M13 (comprising the nucleotide sequence of SEQ ID NO. 45), marker M8 (comprising the nucleotide sequence of SEQ ID NO. 40), marker M9 (comprising the nucleotide sequence of SEQ ID NO. 41), marker M10 (comprising the nucleotide sequence of SEQ ID NO. 42), marker M14 (comprising the nucleotide sequence of SEQ ID NO. 46), marker M15 (comprising the nucleotide sequence of SEQ ID NO. 47) or marker M16 (comprising the nucleotide sequence of SEQ ID NO. 48).

Yet another method for producing a Beta vulgaris plant with optimally fitted large subunit and one or more regulatory subunits of an ALS holoenzyme is described, comprising the steps of providing a Beta vulgaris plant comprising an allele encoding a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan, such as an amino acid sequence of SEQ ID NO. 3; and adapting, by genome-editing or directed mutation, the nucleotide sequence of the allele on chromosome 3 and/or the allele on chromosome 4 encoding a small subunit of ALS to obtain a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.

Genome editing or targeted editing techniques refers to any method, protocol, or technique that allows the precise and/or targeted editing of a specific location in a genome (e.g., the editing is not random). Without being limiting, use of a site-specific nuclease is one example of a targeted editing technique.

As used herein, “editing” or “genome editing” refers to targeted mutagenesis, insertion, deletion, or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence.

Genome editing or targeted editing can be effected via the use of one or more site-specific nucleases. Site-specific nucleases can induce a double-stranded break (DSB) at a target site of a genome sequence that is then repaired by the natural processes of either homologous recombination (HR) or non-homologous end-joining (NHEJ). Sequence modifications, such as insertions, deletions, can occur at the DSB locations via NHEJ repair. HR can be used to integrate a donor nucleic acid sequence into a target site. If two DSBs flanking one target region are created, the breaks can be repaired via NHEJ by reversing the orientation of the targeted DNA (also referred to as an “inversion”).

Site-specific nucleases provided herein can be used as part of a targeted editing technique. Non-limiting examples of site-specific nucleases used in methods and/or compositions provided herein include meganucleases, zinc finger nucleases (ZFNs), transcription activator-like effector nucleases (TALENs), RNA-guided nucleases (e.g., Cas9 and Cpfl), a recombinase (without being limiting, for example, a serine recombinase attached to a DNA recognition motif, a tyrosine recombinase attached to a DNA recognition motif), a transposase (without being limiting, for example, a DNA transposase attached to a DNA binding domain), or any combination thereof. In one aspect, a method provided herein comprises the use of one or more, two or more, three or more, four or more, or five or more site-specific nucleases to induce one, two, three, four, five, or more than five DSBs at one, two, three, four, five, or more than five target sites.

An RNA guided nuclease can be selected from the group consisting of a Cas9 or a Cpfl.

In another aspect a site-specific nuclease provided herein is selected from the group consisting of a Casl, a CaslB, a Cas2, a Cas3, a Cas4, a Cas5, a Cas6, a Cas7, a Cas8, a Cas9, a Casl0, a Csyl, a Csy2, a Csy3, a Csel, a Cse2, a Cscl, a Csc2, a Csa5, a Csn2, a Csm2, a Csm3, a Csm4, a Csm5, a Csm6, a Cmrl, a Cmr3, a Cmr4, a Cmr5, a Cmr6, a Csbl, a Csb2, a Csb3, a Csxl7, a Csxl4, a CsxlO, a Csxl6, a CsaX, a Csx3, a Csxl, a Csxl5, a Csfl, a Csf2, a Csf3, a Csf4, a Cpfl, a homolog thereof, or a modified version thereof. In another aspect, an RNA-guided nuclease provided herein is selected from the group consisting of a Cas9 or a Cpfl. In another aspect an RNA guided nuclease provided herein is selected from the group consisting of a Casl, a CaslB, a Cas2, a Cas3, a Cas4, a Cas5, a Cas6, a Cas7, a Cas8, a Cas9, a Casl0, a Csyl, a Csy2, a Csy3, a Csel, a Cse2, a Cscl, a Csc2, a CsaS, a Csn2, a Csm2, a Csm3, a Csm4, a CsmS, a Csm6, a Cmrl, a Cmr3, a Cmr4, a Cmr5, a Cmr6, a Csbl, a Csb2, a Csb3, a Csxl7, a Csxl4, a CsxlO, a Csxl6, a CsaX, a Csx3, a Csxl, a Csxl5, a Csfl, a Csf2, a Csf3, a Csf4, a Cpfl, a homolog thereof, or a modified version thereof.

RNA guided nucleases require the presence of guide RNAs and/or tracr RNAs targeted to the nucleic acid of interest. Design of such guide RNAs or single guide RNAs is well established in the art.

In some jurisdictions, plant products obtained exclusively by essentially biological processes may be excluded from patentability. “Exclusively obtained by essentially biological processes” refers to products obtained by processes which do not require any technical or human intervention. Essentially biological processes refer to processes which consist entirely of natural phenomena, such as crossing or selecting. In one particular embodiment, the invention may be related to Beta vulgaris plants as herein described which are not exclusively obtained by essentially biological processes, more particularly are not obtained by processes which consist entirely of natural phenomena, such as crossing or selecting.

In another aspect of the invention, use of one or more one or more ALS inhibitor herbicide(s) for controlling unwanted vegetation in Beta vulgaris growing areas wherein the Beta vulgaris plants are Beta vulgaris plants as herein described, is provided.

The ALS inhibitor herbicides may belong to any one of those listed in the above numbered embodiments of the invention The “CAS RN” stated in square brackets behind the names (common names) mentioned under groups A to C corresponds to the “chemical abstract service registry number”, a customary reference number which allows the substances named to be classified unambiguously, since the “CAS RN” distinguishes, inter alia, between isomers including stereoisomers. The listed compounds moreover are further indicated by a number between brackets, such as A1-1 etc., used further hereinafter.

In the context of the invention, “tolerance” or “tolerant” means that the application of one or more ALS inhibitor herbicide(s) belonging to any of the above defined groups (A), (B), (C) does not show any apparent effect(s) concerning the physiological functions/phytotoxicity when applied to the Beta vulgaris plant, especially sugar beet or fodder beet, as herein described and wherein the application of the same amount of the respective ALS inhibitor herbicide(s) on non-tolerant Beta vulgaris plants leads to significant negative effects concerning plant growth, its physiological functions or shows phytotoxic symptoms. Quality and quantity of the observed effects may depend on the chemical composition of the respective ALS inhibitor herbicide(s) applied, dose rate and timing of the application as well growth conditions/stage of the treated plants.

A useful ALS inhibitor herbicide comprises foramsulfuron [CAS RN 173159-57-4] (=A1-13) and thiencarbazone-methyl [CAS RN 317815-83-1] (=A2-3).

Another ALS inhibitor herbicide which may be used for control of unwanted vegetation in Beta vulgaris (preferably sugar beet) growing areas in which the Beta vulgaris (preferably sugar beet) plants are B. vulgaris plants as herein described is imazamox [CAS RN 114311-32-9] (=B1-2).

Another ALS inhibitor herbicide which may be used for control of unwanted vegetation in Beta vulgaris (preferably sugar beet) growing areas in which the Beta vulgaris (preferably sugar beet) plants are B. vulgaris plants as herein described is bispyribac-sodium [CAS RN 125401-92-5] (=C1-1).

Another ALS inhibitor herbicide which may be used for control of unwanted vegetation in Beta vulgaris (preferably sugar beet) growing areas in which the Beta vulgaris (preferably sugar beet) plants are B. vulgaris plants as herein described is triflusulfuron-methyl.

Additionally, the ALS inhibitor herbicide(s) to be used on B. vulgaris plants as herein described may comprise further components, for example agrochemically active compounds of a different type of mode of action and/or the formulation auxiliaries and/or additives customary in crop protection, or may be used together with these.

In a preferred embodiment, the herbicide combinations to be used according to the invention comprise effective amounts of the ALS inhibitor herbicide(s) belonging to groups (A), (B) and/or (C) and/or have synergistic actions. The synergistic actions can be observed, for example, when applying one or more ALS inhibitor herbicide(s) belonging to groups (A), (B), and/or (C) together, for example as a coformulation or as a tank mix; however, they can also be observed when the active compounds are applied at different times (splitting). It is also possible to apply the herbicides or the herbicide combinations in a plurality of portions (sequential application), for example pre-emergence applications followed by post-emergence applications or early postemergence applications followed by medium or late post-emergence applications.

Preference is given here to the joint or almost simultaneous application of the ALS inhibitor herbicides belonging to groups (A), (B) and/or (C) of the combination in question.

The synergistic effects permit a reduction of the application rates of the individual ALS inhibitor herbicides, a higher efficacy at the same application rate, the control of species which were as yet uncontrolled (gaps), control of species which are tolerant or resistant to individual ALS inhibitor herbicides or to a number of ALS inhibitor herbicides, an extension of the period of application and/or a reduction in the number of individual applications required and—as a result for the user—weed control systems which are more advantageous economically and ecologically.

The herbicides to be used according to this invention are all acetolactate synthase (ALS) inhibitor herbicides (which might alternatively and interchangeably also be named as “ALS inhibiting herbicides”) and thus inhibit protein biosynthesis in plants.

The application rate of the ALS inhibitor herbicides belonging to groups (A), (B) or (C) (as defined above) can vary within a wide range, for example between 0.001 g and 1500 g of ai/ha (ai/ha means here and below “active substance per hectare”=based on 100% pure active compound). Applied at application rates of from 0.001 g 20 to 1500 g of ai/ha, the herbicides belonging to classes A, Band C according to this invention, preferably the compounds A1-1; A1-4; A1-8; A1-9; A1-12; A1-13; A1-16; A1-17; A1-18; A1-19; A1-20; A1-28; A1-29; A1-31; A1-39; A1-41; A1-83; A1-87; A2-2; A2-3; A3-3; A3-5; A3-7, A4-3, control, when used by the pre- and post-emergence method, a relatively wide spectrum of harmful plants, for example of annual and perennial mono- or dicotyledonous weeds, and also of unwanted crop plants (together also defined as “unwanted vegetation).

In many applications according to the invention, the application rates are generally lower, for example in the range of from 0.001 g to 1000 g of ai/ha, preferably from 30 0.1 g to 500 g of ai/ha, particularly preferably from 0.5 g to 250 g of ai/ha, and even more preferably 1.0 g to 200 g of ai/ha. In cases where the application of several ALS inhibitor herbicides is conducted, the quantity represents the total quantity of all of the applied ALS inhibitor herbicides.

For example, the combinations according to the invention of ALS inhibitor herbicides (belonging to groups (A), (B) and/or (C)) allow the activity to be enhanced synergistically in a manner which, by far and in an unexpected manner, exceeds the activities which can be achieved using the individual ALS inhibitor herbicides (belonging to groups (A), (B) and/or (C)). For combinations of ALS inhibitor herbicides, the preferred conditions are illustrated below.

Of particular interest according to present invention is the use of herbicidal compositions for control of unwanted vegetation in Beta vulgaris plants, preferably in sugar beet or fodder beet plants having a content of the following ALS inhibitor herbicides:
(A1-1)+(A1-4); (A1-1)+(A1-8); (A1-1)+(A1-9); (A1-1)+(A1-12);
(A1-1)+(A1-13); (A1-1)+(A1-16); (A1-1)+(A1-17); (A1-1)+(A1-18);
(A1-1)+(A1-19); (A1-1)+(A1-20); (A1-1)+(A1-28); (A1-1)+(A1-29);
(A1-1)+(A1-31); (A1-1)+(A1-39); (A1-1)+(A1-41); (A1-1)+(A1-83);
(A1-1)+(A1-87); (A1-1)+(A2-2); (A1-1)+(A2-3); (A1-1)+(A3-3);
(A1-1)+(A3-5); (A1-1)+(A3-7); (A1-1)+(A4-1); (A1-1)+(A4-2); (A1-1)+(A4-3);
(A1-4)+(A1-8); (A1-4)+(A1-9); (A1-4)+(A1-12); (A1-4)+(A1-13);
(A1-4)+(A1-16); (A1-4)+(A1-17); (A1-4)+(A1-18); (A1-4)+(A1-19);
(A1-4)+(A1-20); (A1-4)+(A1-28); (A1-4)+(A1-29); (A1-4)+(A1-31);
(A1-4)+(A1-39); (A1-4)+(A1-41); (A1-4)+(A1-83); (A1-4)+(A1-87);
(A1-4)+(A2-2); (A1-4)+(A2-3); (A1-4)+(A3-3); (A1-4)+(A3-5);
(A1-4)+(A3-7); (A1-4)+(A4-1); (A1-4)+(A4-2); (A1-4)+(A4-3);
(A1-8)+(A1-9); (A1-8)+(A1-12); (A1-8)+(A1-13); (A1-8)+(A1-16);
(A1-8)+(A1-17); (A1-8)+(A1-18); (A1-8)+(A1-19); (A1-8)+(A1-20);
(A1-8)+(A1-28); (A1-8)+(A1-29); (A1-8)+(A1-31); (A1-8)+(A1-39);
(A1-8)+(A1-41); (A1-8)+(A1-83); (A1-8)+(A1-87); (A1-8)+(A2-2);
(A1-8)+(A2-3); (A1-8)+(A3-3); (A1-8)+(A3-5); (A1-8)+(A3-7);
A1-8)+(A4-1); (A1-8)+(A4-2); (A1-8)+(A4-3);
(A1-9)+(A1-12); (A1-9)+(A1-13); (A1-9)+(A1-16); (A1-9)+(A1-17);
(A1-9)+(A1-18); (A1-9)+(A1-19); (A1-9)+(A1-20); (A1-9)+(A1-28);
(A1-9)+(A1-29); (A1-9)+(A1-31); (A1-9)+(A1-39); (A1-9)+(A1-41);
(A1-9)+(A1-83); (A1-9)+(A1-87); (A1-9)+(A2-2); (A1-9)+(A2-3);
(A1-9)+(A3-3); (A1-9)+(A3-5); (A1-9)+(A3-7); (A1-9)+(A4-1);
(A1-9)+(A4-2); (A1-9)+(A4-3);
(A1-12)+(A1-13); (A1-12)+(A1-16); (A1-12)+(A1-17); (A1-12)+(A1-18);
(A1-12)+(A1-19); (A1-12)+(A1-20); (A1-12)+(A1-28); (A1-12)+(A1-29);
(A1-12)+(A1-31); (A1-12)+(A1-39); (A1-12)+(A1-41); (A1-12)+(A1-83);
(A1-12)+(A1-87); (A1-12)+(A2-2); (A1-12)+(A2-3); (A1-12)+(A3-3);
(A1-12)+(A3-5); (A1-12)+(A3-7); (A1-12)+(A4-1); (A1-12)+(A4-2); (A1-12)+(A4-3);
(A1-13)+(A1-16); (A1-13)+(A1-17); (A1-13)+(A1-18); (A1-13)+(A1-19);
(A1-13)+(A1-20); (A1-13)+(A1-28); (A1-13)+(A1-29); (A1-13)+(A1-31);
(A1-13)+(A1-39); (A1-13)+(A1-41); (A1-13)+(A1-83); (A1-13)+(A1-87);
(A1-13)+(A2-2); (A1-13)+(A2-3); (A1-13)+(A3-3); (A1-13)+(A3-5);
(A1-13)+(A3-7); (A1-13)+(A4-1); (A1-13)+(A4-2); (A1-13)+(A4-3);
(A1-16)+(A1-17); (A1-16)+(A1-18); (A1-16)+(A1-19); (A1-16)+(A1-20);
(A1-16)+(A1-28); (A1-16)+(A1-29); (A1-16)+(A1-31); (A1-16)+(A1-39);
(A1-16)+(A1-41); (A1-16)+(A1-83); (A1-16)+(A1-87); (A1-16)+(A2-2);
30 (A1-16)+(A2-3); (A1-16)+(A3-3); (A1-16)+(A3-5); (A1-16)+(A3-7);
(A1-16)+(A4-1); (A1-16)+(A4-2); (A1-16)+(A4-3);
A1-17)+(A1-18); (A1-17)+(A1-19); (A1-17)+(A1-20); (A1-17)+(A1-28);
(A1-17)+(A1-29); (A1-17)+(A1-31); (A1-17)+(A1-39); (A1-17)+(A1-41);
(A1-17)+(A1-83); (A1-17)+(A1-87); (A1-17)+(A2-2); (A1-17)+(A2-3);
(A1-17)+(A3-3); (A1-17)+(A3-5); (A1-17)+(A3-7); (A1-17)+(A4-1);
(A1-17)+(A4-2); (A1-17)+(A4-3);
(A1-18)+(A1-19); (A1-18)+(A1-20); (A1-18)+(A1-28); (A1-18)+(A1-29);
(A1-18)+(A1-31); (A1-18)+(A1-39); (A1-18)+(A1-41); (A1-18)+(A1-83);
(A1-18)+(A1-87); (A1-18)+(A2-2); (A1-18)+(A2-3); (A1-18)+(A3-3);
(A1-18)+(A3-5); (A1-18)+(A3-7); (A1-18)+(A4-1); (A1-18)+(A4-2);
(A1-18)+(A4-3);
(A1-19)+(A1-20); (A1-19)+(A1-28); (A1-19)+(A1-29); (A1-19)+(A1-31);
(A1-19)+(A1-39); (A1-19)+(A1-41); (A1-19)+(A1-83); (A1-19)+(A1-87);
(A1-19)+(A2-2); (A1-19)+(A2-3); (A1-19)+(A3-3); (A1-19)+(A3-5);
(A1-19)+(A3-7); (A1-19)+(A4-1); (A1-19)+(A4-2); (A1-19)+(A4-3);
(A1-20)+(A1-28); (A1-20)+(A1-29); (A1-20)+(A1-31); (A1-20)+(A1-39);
(A1-20)+(A1-41); (A1-20)+(A1-83); (A1-20)+(A1-87); (A1-20)+(A2-2);
(A1-20)+(A2-3); (A1-20)+(A3-3); (A1-20)+(A3-5); (A1-20)+(A3-7);
(A1-20)+(A4-1); (A1-20)+(A4-2); (A1-20)+(A4-3);
(A1-28)+(A1-29); (A1-28)+(A1-31); (A1-28)+(A1-39); (A1-28)+(A1-41);
(A1-28)+(A1-83); (A1-28)+(A1-87); (A1-28)+(A2-2); (A1-28)+(A2-3);
(A1-28)+(A3-3); (A1-28)+(A3-5); (A1-28)+(A3-7); (A1-28)+(A4-1);
(A1-28)+(A4-2); (A1-28)+(A4-3);
(A1-29)+(A1-31); (A1-29)+(A1-39); (A1-29)+(A1-41); (A1-29)+(A1-83);
(A1-29)+(A1-87); (A1-29)+(A2-2); (A1-29)+(A2-3); (A1-29)+(A3-3);
(A1-29)+(A3-5); (A1-29)+(A3-7); (A1-29)+(A4-1); (A1-29)+(A4-2); (A1-29)+(A4-3);
(A1-31)+(A1-39); (A1-31)+(A1-41); (A1-31)+(A1-83); (A1-31)+(A1-87);
(A1-31)+(A2-2); (A1-31)+(A2-3); (A1-31)+(A3-3); (A1-31)+(A3-5);
(A1-31)+(A3-7); (A1-31)+(A4-1); (A1-31)+(A4-2); (A1-31)+(A4-3);
(A1-39)+(A1-41); (A1-39)+(A1-83); (A1-39)+(A1-87); (A1-39)+(A2-2);
(A1-39)+(A2-3); (A1-39)+(A3-3); (A1-39)+(A3-5); (A1-39)+(A3-7);
(A1-39)+(A4-1); (A1-39)+(A4-2); (A1-39)+(A4-3);
(A1-41)+(A1-83); (A1-41)+(A1-87); (A1-41)+(A2-2); (A1-41)+(A2-3);
(A1-41)+(A3-3); (A1-41)+(A3-5); (A1-41)+(A3-7); (A1-41)+(A4-1);
(A1-41)+(A4-2); (A1-41)+(A4-3);
(A1-83)+(A2-2); (A1-83)+(A2-3); (A1-83)+(A3-3); (A1-83)+(A3-5);
(A1-83)+(A3-7); (A1-83)+(A4-1); (A1-83)+(A4-2); (A1-83)+(A4-3);
(A1-87)+(A2-2); (A1-87)+(A2-3); (A1-87)+(A3-3); (A1-87)+(A3-5);
(A1-87)+(A3-7); (A1-87)+(A4-1); (A1-87)+(A4-2); (A1-87)+(A4-3);
(A2-2)+(A2-3); (A2-2)+(A3-3); (A2-2)+(A3-5); (A2-2)+(A3-7);
(A2-2)+(A4-1); (A2-2)+(A4-2); (A2-2)+(A4-3);
(A2-3)+(A3-3); (A2-3)+(A3-5); (A2-3)+(A3-7);
(A2-3)+(A4-1); (A2-3)+(A4-2); (A2-3)+(A4-3);
(A3-3)+(A3-5); (A3-3)+(A3-7);
(A3-3)+(A4-1); (A3-3)+(A4-2); (A3-3)+(A4-3);
(A3-5)+(A3-7); (A3-5)+(A4-1); (A3-5)+(A4-2); (A3-5)+(A4-3);
(A3-7)+(A4-1); (A3-7)+(A4-2); (A3-7)+(A4-3);
(A-1)+(A4-2); (A4-1)+(A4-3); and
(A4-2)+(A4-3).

Additionally, the ALS inhibitor herbicides to be used according to the invention may comprise further components, for example agrochemically active compounds of a different type of mode of action and/or the formulation auxiliaries and/or additives customary in crop protection, or may be used together with these.

The ALS inhibitor herbicide(s) to be used according to the invention or combinations of various such ALS inhibitor herbicides may furthermore comprise various agrochemically active compounds, for example from the group of the safeners, fungicides, insecticides, or from the group of the formulation auxiliaries and additives customary in crop protection.

In a further embodiment, the invention relates to the use of effective amounts of ALS inhibitor herbicide(s) (i.e. members of the groups (A), (B) and/or (C)) and non-ALS inhibitor herbicides (i.e. herbicides showing a mode of action that is different to the inhibition of the ALS enzyme [acetohydroxyacid synthase; EC 2.2.1.6] (group D herbicides) in order obtain synergistic effect for the control of unwanted vegetation.

Such synergistic actions can be observed, for example, when applying one or more ALS inhibitor herbicides (i.e. members of the groups (A), (B), and/or (C)) and one or more non-ALS inhibitor herbicides (group D herbicides) together, for example as a coformulation or as a tank mix; however, they can also be observed when the active compounds are applied at different times (splitting). It is also possible to apply the ALS inhibitor herbicides and non-ALS inhibitor herbicides in a plurality of portions (sequential application), for example pre-emergence applications followed by post-emergence applications or early post-emergence applications followed by medium or late post-emergence applications. Preference is given here to the joint or almost simultaneous application of the herbicides ((A), (B) and/or (C)) and (D) of the combination in question.

Suitable partner herbicides to be applied together with ALS inhibitor herbicides are, for example, the following herbicides which differ structurally from the herbicides belonging to the groups (A), (B), and (C) as defined above, preferably herbicidally active compounds whose action is based on inhibition of, for example, acetyl coenzyme A carboxylase, PSI, PSII, HPPDO, phytoene desaturase, protoporphyrinogen oxidase, glutamine synthetase, cellulose biosynthesis, 5-enol-pyruvyl-shikimate 3-phosphate synthetase, as described, for example, in Weed Research 26, 441-445 (1986), or “The Pesticide Manual”, 14th edition, The British Crop Protection Council, 2007, or 15th edition 2010, or in the corresponding “ePesticide Manual”, Version 5 (2010), in each case published by the British Crop Protection Council, (hereinbelow in short also “PM”), and in the literature cited therein. Lists of common names are also available in “The Compendium of Pesticide Common Names” on the internet. Herbicides known from the literature (in brackets behind the common name hereinafter also classified by the indicators D1 to D426), which can be combined with ALS-inhibitor herbicides of groups (A), (B) and/or (C) and to be used according to present invention are, for example, the active compounds listed below: (note: the herbicides are referred to either by the “common name” in accordance with the International Organization for Standardization (ISO) or by the chemical name, together where appropriate with a customary code number, and in each case include all use forms, such as acids, salts, esters and isomers, such as stereoisomers and optical isomers, in particular the commercial form or the commercial forms, unless the context indicates otherwise. The citation given is of one use form and in some cases of two or more use forms): acetochlor (=D1), acibenzolar (=D2), acibenzolar-S-methyl (=D3), acifluorfen (=D4), acifluorfen-sodium (=D5), aclonifen (=D6), alachlor (=D7), allidochlor (=D8), alloxydim (=D9), alloxydim-sodium (=D10), ametryn (=D11), amicarbazone (=D12), amidochlor (=D13), aminocyclopyrachlor (=D14), aminopyralid (=D15), amitrole (=D16), ammonium sulfamate (=D17), ancymidol (=D18), anilofos (=D19), asulam (=D20), atrazine (=D21), azafenidin (=D22), aziprotryn (=D23), beflubutamid (=D24), benazolin (=D25), benazolin-ethyl (=D26), bencarbazone (=D27), benfluralin (=D28), benfuresate (=D29), bensulide (=D30), bentazone (=D31), benzfendizone (=D32), benzobicyclon (=D33), benzofenap (=D34), benzofluor (=D35), benzoylprop (=D36), bicyclopyrone (=D37), bifenox (=D38), bilanafos (=D39), bilanafos-sodium (=D40), bromacil (=D41), bromobutide (=D42), bromofenoxim (=D43), bromoxynil (=D44), bromuron (=D45), buminafos (=D46), busoxinone (=D47), butachlor (=D48), butafenacil (=D49), butamifos (=D50), butenachlor (=D51), butralin (=D52), butroxydim (=D53), butylate (=D54), cafenstrole (=D55), carbetamide (=D56), carfentrazone (=D57), carfentrazoneethyl (=D58), chlomethoxyfen (=D59), chloramben (=D60), chlorazifop (=D61), chlorazifop-butyl (=D62), chlorbromuron (=D63), chlorbufam (=D64), chlorfenac (=D65), chlorfenac-sodium (=D66), chlorfenprop (=D67), chlorflurenol (=D68), chlorflurenol-methyl (=D69), chloridazon (=D70), chlormequat-chloride (=D71), chlornitrofen (=D72), chlorophthalim (=D73), chlorthal-dimethyl (=D74), chlorotoluron (=D75), cinidon (=D76), cinidon-ethyl (=D77), cinmethylin (=D78), clethodim (=D79), clodinafop (=D80), clodinafop-propargyl (=D81), clofencet (=D82), clomazone (=D83), clomeprop (=D84), cloprop (=D85), clopyralid (=D86), cloransulam (=D87), cloransulam-methyl (=D88), cumyluron (=D89), cyanamide (=D90), cyanazine (=D91), cyclanilide (=D92), cycloate (=D93), cycloxydim (=D94), cycluron (=D95), cyhalofop (=D96), cyhalofop-butyl (=D97), cyperquat (=D98), cyprazine (=D99), cyprazole (=D100), 2,4-D (=D101), 2,4-DB (=D102), daimuron/dymron (=D103), dalapon (=D104), daminozide (=D105), dazomet (=D106), n-decanol (=D-107), desmedipham (=D108), desmetryn (=D109), detosyl-pyrazolate (=D110), diallate (=D111), dicamba (=D112), dichlobenil (=D113), dichlorprop (=D114), dichlorprop-P (=D115), diclofop (=D116), diclofop-methyl (=D117), diclofop-P-methyl (=D118), diethatyl (=D119), diethatyl-ethyl (=D120), difenoxuron (=D121), difenzoquat (=D122), diflufenican (=D123), diflufenzopyr (=D124), diflufenzopyr-sodium (=D125), dimefuron (=D126), dikegulac-sodium (=D127), dimefuron (=D128), dimepiperate (=D129), dimethachlor (=D130), dimethametryn (=D131), dimethenamid (=D132), dimethenamid-P (=D133), dimethipin (=D134), dimetrasulfuron (=D135), dinitramine (=D136), dinoseb (=D137), dinoterb (=D138), diphenamid (=D139), dipropetryn (=D140), diquat (=D141), diquat-dibromide (=D142), dithiopyr (=D143), diuron (=D144), DNOC (=D145), eglinazine-ethyl (=D146), endothall (=D147), EPTC (=D148), esprocarb (=D149), ethalfluralin (=D150), ethephon (=D151), ethidimuron (=D152), ethiozin (=D153), ethofumesate (=D154), ethoxyfen (=D155), ethoxyfen-ethyl (=D156), etobenzanid (=D157), F-5331 (=2-Chlor-4-fluor-5-[4-(3-fluorpropyl)-4, 5-dihydro-5-oxo-1H-tetrazol-1-yl]-phenyl]ethansulfonamid) (=D158), F-7967 (=3-[7-Chlor-5-fluor-2-(trifluormethyl)-1H-10 benzimidazol-4-yl]-1-methyl-6-(trifluormethyl)pyrimidin-2,4(1H,3H)-dion) (=D159), fenoprop (=D160), fenoxaprop (=D161), fenoxaprop-P (=D162), fenoxaprop-ethyl (=D163), fenoxaprop-P-ethyl (=D164), fenoxasulfone (=D165), fentrazamide (=D166), fenuron (=D167), flam prop (=D168), flamprop-M-isopropyl (=D169), flamprop-M-methyl (=D170), fluazifop (=D171), fluazifop-P (=D172), fluazifop-butyl 15 (=D173), fluazifop-P-butyl (=D174), fluazolate (=D175), fluchloralin (=D176), flufenacet (thiafluamide) (=D177), flufenpyr (=D178), flufenpyr-ethyl (=D179), flumetralin (=D180), flumiclorac (=D181), flumiclorac-pentyl (=D182), flumioxazin (=D183), flumipropyn (=D184), fluometuron (=D185), fluorodifen (=D186), fluoroglycofen (=D187), fluoroglycofen-ethyl (=D188), flupoxam (=D189), 20 flupropacil (=D190), flupropanate (=D191), flurenol (=D192), flurenol-butyl (=D193), fluridone (=D194), flurochloridone (=D195), fluroxypyr (=D196), fluroxypyr-meptyl (=D197), flurprimidol (=D198), flurtamone (=D199), fluthiacet (=D200), fluthiacet-methyl (=D201), fluthiamide (=D202), fomesafen (=203), forchlorfenuron (=D204), fosamine (=D205), furyloxyfen (=D206), gibberellic acid 25 (=D207), glufosinate (=D208), glufosinate-ammonium (=D209), glufosinate-P (=D210), glufosinate-P-ammonium (=D211), glufosinate-P-sodium (=D212), glyphosate (=D213), glyphosate-isopropylammonium (=D214), H-9201 (=O-(2,4-Dimethyl-6-nitrophenyl)-O-ethyl-isopropylphosphoramidothioat) (=D215), halosafen (=D216), haloxyfop (=D217), haloxyfop-P (=D218), haloxyfop-ethoxyethy (=D219), haloxyfop-P-ethoxyethyl (=D220), haloxyfop-methyl (=D221), haloxyfopP-methyl (=D222), hexazinone (=D223), HW-02 (=1-(Dimethoxyphosphoryl)-ethyl(2,4-dichlorphenoxy)acetate) (=D224), inabenfide (=D225), indanofan (=D226), indaziflam (=D227), indol-3-acetic acid (IAA) (=D228), 4-indol-3-ylbutyricacid (IBA) (=D229), ioxynil (=D230), ipfencarbazone (=D231), isocarbamid (=D232), isopropalin (=D233), isoproturon (=D234), isouron (=D235), isoxaben (=D236), isoxachlortole (=D237), isoxaflutole (=D238), isoxapyrifop (=D239), KUH-043 (=3-({[5-(Difluormethyl)-1-methyl-3-(trifluormethyl)-1H-pyrazol-4-yl]methyl}sulfonyl)-5,5-dimethyl-4,5-dihydro-1,2-oxazol) (=D240), karbutilate (=D241), ketospiradox (=D242), lactofen (=D243), lenacil (=D244), linuron (=D245), maleic hydrazide (=D246), MCPA (=D247), MCPB (=D248), MCPB-methyl, -ethyl and -sodium (=D249), mecoprop (=D250), mecoprop-sodium (=D251), mecoprop-butotyl (=D252), mecoprop-P-butotyl (=D253), mecoprop-P-dimethylammonium (=D254), mecoprop-P-2-ethylhexyl (=D255), mecoprop-P-potassium (=D256), mefenacet (=D257), mefluidide (=D258), mepiquat-chloride (=D259), mesotrione (=D260), methabenzthiazuron (=D261), metam (=D262), metamifop (=D263), metamitron (=D264), metazachlor (=D265), metazole (=D266), methiopyrsulfuron (=D267), methiozolin (=D268), methoxyphenone (=D269), methyldymron (=D270), 1-methylcyclopropen (=D271), methylisothiocyanat (=D272), metobenzuron (=D273), metobromuron (=D274), metolachlor (=D275), S-metolachlor (=D-276), metoxuron (=D277), metribuzin (=D278), molinate (=D279), monalide (=D280), monocarbamide (=D281), monocarbamide-dihydrogensulfate (=D282), monolinuron (=D283), monosulfuronester (=D284), monuron (=D285), MT-128 (=6-Chlor-N-[(2E)-3-chlorprop-2-en-1-yl]-5-methyl-N-phenylpyridazin-3-amine) (=D286), MT-5950 (=N-[3-Chlor-4-(1-methylethyl)-phenyl]-2-methylpentanamide) (=D287), NGGC-011 (=D288), naproanilide (=D289), napropamide (=D290), naptalam (=D291), NC-310 (=4-(2,4-Dichlorobenzoyl)-1-methyl-5-benzyloxypyrazole) (=D292), neburon (=D293), nipyraclofen (=D294), nitralin (=D295), nitrofen (=D296), nitrophenolat-sodium (isomer mixture) (=D297), nitrofluorfen (=D298), nonanoic acid (=D299), norflurazon (=D300), orbencarb (=D301), oryzalin (=D302), oxadiargyl (=D303), oxadiazon (=D304), oxaziclomefone (=D305), oxyfluorfen (=D306), paclobutrazol (=D307), paraquat (=D308), paraquat-dichloride (=D309), pelargonic acid (nonanoic acid) (=D310), pendimethalin (=D311), pendralin (=D312), pentanochlor (=D313), pentoxazone (=D314), perfluidone (=D315), pethoxamid (=D317), phenisopham (=D318), phenmedipham (=D319), phenmedipham-ethyl (=D320), picloram (=D321), picolinafen (=D322), pinoxaden (=D323), piperophos (=D324), pirifenop (=D325), pirifenop-butyl (=D326), pretilachlor (=D327), probenazole (=D328), profluazol (=D329), procyazine (=D330), prodiamine (=D331), prifluraline (=D332), profoxydim (=D333), prohexadione (=D334), prohexadionecalcium (=D335), prohydrojasmone (=D336), prometon (=D337), prometryn (=D338), propachlor (=D339), propanil (=D340), propaquizafop (=D341), propazine (=D342), propham (=D343), propisochlor (=D344), propyzamide (=D345), prosulfalin (=D346), prosulfocarb (=D347), prynachlor (=D348), pyraclonil (=D349), pyraflufen (=D350), pyraflufen-ethyl (=D351), pyrasulfotole (=D352), pyrazolynate (pyrazolate) (=D353), pyrazoxyfen (=D354), pyribambenz (=D355), pyributicarb (=D356), pyridafol (=D357), pyridate (=D358), pyriminobac (=D359), 15 pyrimisulfan (=D360), pyroxasulfone (=D361), quinclorac (=D362), quinmerac (=D363), quinoclamine (=D364), quizalofop (=D365), quizalofop-ethyl (=D366), quizalofop-P (=D367), quizalofop-P-ethyl (=D368), quizalofop-P-tefuryl (=D369), saflufenacil (=D370), secbumeton (=D371), sethoxydim (=D372), siduron (=D373), simazine (=D374), simetryn (=D375), SN-106279 (=Methyl-(2R)-2-({7-20 [2-chlor-4-(trifluormethyl)phenoxy]-2-naphthyl}oxy)-propanoate) (=D376), sulcotrione (=D377), sulfallate (CDEC) (=D378), sulfentrazone (=D379), sulfosate (glyphosate-trimesium) (=D380), SYN-523 (=D381), SYP-249 (=1-Ethoxy-3-methyl-1-oxobut-3-en-2-yl-5-[2-chlor-4-(trifluormethyl)phenoxy]-2-nitrobenzoate) (=D382), tebutam (=D383), tebuthiuron (=D384), teenazene (=D385), tefuryltrione (=D386), tembotrione (=D387), tepraloxydim (=D388), terbacil (=D389), terbucarb (=D390), terbuchlor (=D391), terbumeton (=D392), terbuthylazine (=D393), terbutryn (=D394), thenylchlor (=D395), thiafluamide (=D396), thiazafluron (=D397), thiazopyr (=D398), thidiazimin (=D399), thidiazuron (=D400), thiobencarb (=D401), tiocarbazil (=D402), topramezone (=D403), tralkoxydim (=D404), triallate (=D405), triaziflam (=D406), triazofenamide (=D407), trichloracetic acid (TCA) (=D408), triclopyr (=D409), tridiphane (=D410), trietazine (=D411), trifluralin (=D412), trimeturon (=D413), trinexapac (=D414), trinexapac-ethyl (=D415), tsitodef (=D416), uniconazole (=D417), uniconazole-P (=D418), vernolate (=D419), ZJ-0862 (=3,4-Dichlor-N-{2-[(4,6-dimethoxypyrimidin-2-yl)oxy]benzyl}aniline) (=D420), and the below compounds defined by their chemical structure, respectively

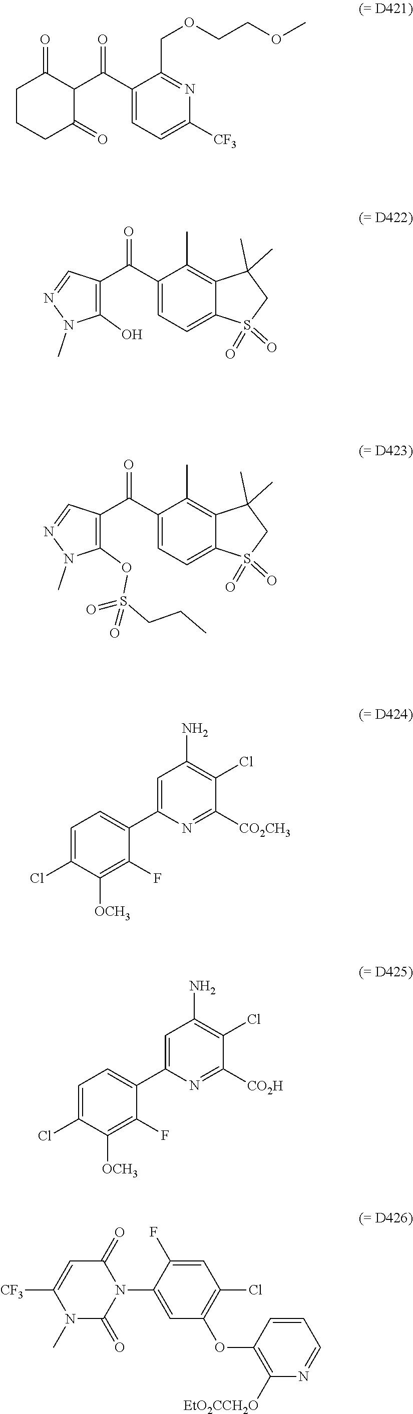

Preferably, further herbicides which differ structurally and via their mode of action from the ALS inhibitor herbicides belonging to the groups (A), (B), and (C) as defined above and to be applied according to the present invention for control of unwanted vegetation in ALS inhibitor herbicide tolerant Beta vulgaris plants, preferably sugar beet or fodder beet plants as herein described. [0064]. In connection with ALS inhibitor herbicides belonging to the groups (A), (B), and (C) are those belonging to the group of: chloridazon (=D70), clethodim (=D79), clodinafop (=D80), clodinafop-propargyl (=D81), clopyralid (=D86), cycloxydim (=D94), desmedipham (=D108), dimethenamid (=D132), dimethenamid-P (=D133), ethofumesate (=D154), fenoxaprop (=D161), fenoxaprop-P (=D162), fenoxaprop-ethyl (=D163), fenoxaprop-P-ethyl (=D164), fluazifop (=D171), fluazifop-P (=D172), fluazifopbutyl (=D173), fluazifop-P-butyl (=D174), glufosinate (=D208), glufosinateammonium (=D209), glufosinate-P (=D210), glufosinate-P-ammonium (=D211), glufosinate-P-sodium (=D212), glyphosate (=D213), glyphosate-isopropylammonium (=D214), haloxyfop (=D217), haloxyfop-P (=D218), haloxyfopethoxyethyl (=D219), haloxyfop-P-ethoxyethyl (=D220), haloxyfop-methyl (=D221), haloxyfop-P-methyl (=D222), lenacil (=D244), metamitron (=D264), phenmedipham (=D319), phenmedipham-ethyl (=D320), propaquizafop (=D341), quinmerac (=D363), quizalofop (=D365), quizalofop-ethyl (=D366), quizalofop-P (=D367), quizalofop-P-ethyl (=D368), quizalofop-P-tefuryl (=D369), sethoxydim (=D372)

Even more preferably, further herbicides which differ from the ALS inhibitor herbicides belonging to the groups (A), (B), and (C) as defined above and to be applied according to the invention in connection with ALS inhibitor herbicides belonging to the groups (A), (B), and (C) are those belonging to the group of: desmedipham (=D108), ethofumesate (=D154), glufosinate (=D208), glufosinateammonium (=D209), glufosinate-P (=D210), glufosinate-P-ammonium (=D211), glufosinate-P-sodium (=D212), glyphosate (=D213), glyphosate-isopropylammonium (=D214), lenacil (=D244), metamitron (=D264), phenmedipham (=D319), phenmedipham-ethyl (=D320).

Mixtures containing ALS inhibitor herbicides and non-ALS inhibitor herbicides, compositions comprising mixtures of one or more ALS inhibitor herbicide(s) (compounds belonging to one or more of groups (A), (B) and (C)) and non-ALS inhibitor heribicide(s) (group (D) members; as defined above) that are of very particular interest in order to be used according to present invention for control of unwanted vegetation are:
(A1-1)+(D108); (A1-1)+(D154); (A1-1)+(D208); (A1-1)+(D209);
(A1-1)+(D210); (A1-1)+(D212); (A1-1)+(D213); (A1-1)+(D214);
(A1-1)+(D244); (A1-1)+(D264); (A1-1)+(D319); (A1-1)+(D320).
(A1-13)+(D108); (A1-13)+(D154); (A1-13)+(D208); (A1-13)+(D209);
(A1-13)+(D210); (A1-13)+(D212); (A1-13)+(D213); (A1-13)+(D214);
(A1-13)+(D244); (A1-13)+(D264); (A1-13)+(D319); (A1-13)+(D320).
(A1-16)+(D108); (A1-16)+(D154); (A1-16)+(D208); (A1-16)+(D209);
(A1-16)+(D210); (A1-16)+(D212); (A1-16)+(D213); (A1-16)+(D214);
(A1-16)+(D244); (A1-16)+(D264); (A1-16)+(D319); (A1-16)+(D320).
(A1-39)+(D108); (A1-39)+(D154); (A1-39)+(D208); (A1-39)+(D209);
(A1-39)+(D210); (A1-39)+(D212); (A1-39)+(D213); (A1-39)+(D214);
(A1-39)+(D244); (A1-39)+(D264); (A1-39)+(D319); (A1-39)+(D320).
(A1-41)+(D108); (A1-41)+(D154); (A1-41)+(D208); (A1-41)+(D209);
(A1-41)+(D210); (A1-41)+(D212); (A1-41)+(D213); (A1-41)+(D214);
(A1-41)+(D244); (A1-41)+(D264); (A1-41)+(D319); (A1-41)+(D320).
(A1-83)+(D108); (A1-83)+(D154); (A1-83)+(D208); (A1-83)+(D209);
(A1-83)+(D210); (A1-83)+(D212); (A1-83)+(D213); (A1-83)+(D214);
(A1-83)+(D244); (A1-83)+(D264); (A1-83)+(D319); (A1-83)+(D320).
(A1-87)+(D108); (A1-87)+(D154); (A1-87)+(D208); (A1-87)+(D209);
(A1-87)+(D210); (A1-87)+(D212); (A1-87)+(D213); (A1-87)+(D214);
(A1-87)+(D244); (A1-87)+(D264); (A1-87)+(D319); (A1-87)+(D320).
(A2-3)+(D108); (A2-3)+(D154); (A2-3)+(D208); (A2-3)+(D209);
(A2-3)+(D210); (A2-3)+(D212); (A2-3)+(D213); (A2-3)+(D214);
(A2-3)+(D244); (A2-3)+(D264); (A2-3)+(D319); (A2-3)+(D320).
(B1-2)+(D108); (B1-2)+(D154); (B1-2)+(D208); (B1-2)+(D209);
(B1-2)+(D210); (B1-2)+(D212); (B1-2)+(D213); (B1-2)+(D214);
(B1-2)+(D244); (B1-2)+(D264); (B1-2)+(D319); (B1-2)+(D320).
(C1-1)+(D108); (C1-1)+(D154); (C1-1)+(D208); (C1-1)+(D209);
(C1-1)+(D210); (C1-1)+(D212); (C1-1)+(D213); (C1-1)+(D214);
(C1-1)+(D244); (C1-1)+(D264); (C1-1)+(D319); (C1-1)+(D320).

The application of ALS inhibitor herbicides also act efficiently on perennial weeds which produce shoots from rhizomes, root stocks and other perennial organs and which are difficult to control. Here, the substances can be applied, for example, by the pre-sowing method, the pre-emergence method or the post-emergence method, for example jointly or separately. Preference is given, for example, to application by the post-emergence method, in particular to the emerged harmful plants.

Specific examples may be mentioned of some representatives of the monocotyledonous and dicotyledonous weed flora which can be controlled by the ALS inhibitor herbicides, without the enumeration being restricted to certain species.

Examples of weed species on which the application according to present invention act efficiently are, from amongst the monocotyledonous weed species, Avena spp., Alopecurus spp., Apera spp., Brachiaria spp., Bromus spp., Digitaria spp., Lolium spp., Echinochloa spp., Panicum spp., Phalaris spp., Paa spp., Setaria spp. And also Cyperus species from the annual group, and, among the perennial species, Agropyron, Cynodon, Imperata and Sorghum and also perennial Cyperus species.

In the case of the dicotyledonous weed species, the spectrum of action extends to genera such as, for example, Abutilon spp., Amaranthus spp., Chenopodium spp., Chrysanthemum spp., Galium spp., Ipomoea spp., Kochia spp., Lamium spp., Matricaria spp., Pharbitis spp., Polygonum spp., Sida spp., Sinapis spp., Solanum spp., Stellaria spp., Veronica spp. and Viola spp., Xanthium spp., among the annuals, and Convolvulus, Cirsium, Rumex and Artemisia in the case of the perennial weeds.

As used herein unless clearly indicated otherwise, the term “plant” is intended to mean a plant at any developmental stage.

The present invention furthermore provides a method for controlling unwanted vegetation in Beta vulgaris plants as herein described, preferably in sugar beet or fodder beet, which comprises applying one or more ALS inhibitor herbicides belonging to groups (A), (B) and/or (C) to the plants (for example harmful plants, such as monocotyledonous or dicotyledonous weeds or unwanted crop plants), the seed (seeds or vegetative propagation organs, such as tubers or shoot parts) or to the area in which the plants grow (for example the area under cultivation), for example together or separately.

The present invention furthermore provides a method for controlling unwanted vegetation in Beta vulgaris plants as herein described, preferably in sugar beet or fodder beet, which comprises applying one or more ALS inhibitor herbicide(s) belonging to groups (A), (B) and/or (C) alone or in combination with non-ALS inhibitor herbicides belonging to class (D) compound according to the invention to the plants (for example harmful plants, such as monocotyledonous or dicotyledonous weeds or unwanted crop plants), the seed (seeds or vegetative propagation organs, such as tubers or shoot parts) or to the area in which the plants grow (for example the area under cultivation), for example together or separately. One or more non-ALS inhibitor herbicides may be applied in combination with one or more ALS inhibitor herbicide(s) before, after or simultaneously with the ALS inhibitor herbicide(s) to the plants, the seed or the area in which the plants grow (for example the area under cultivation).

“Unwanted plants” or “unwanted vegetation” are to be understood as meaning all plants which grow in locations where they are unwanted. This can, for example, be harmful plants (for example monocotyledonous or dicotyledonous weeds or unwanted crop plants).

The herbicide combinations to be used according to the invention can be prepared by known processes, for example as mixed formulations of the individual components, if appropriate with further active compounds, additives and/or customary formulation auxiliaries, which combinations are then applied in a customary manner diluted with water, or as tank mixes by joint dilution of the components, formulated separately or formulated partially separately, with water. Also possible is the split application of the separately formulated or partially separately formulated individual components.

It is also possible to apply ALS inhibitor herbicides or the combination comprising ALS inhibitor herbicide(s) and non-ALS inhibitor herbicide(s) in a plurality of portions (sequential application) using, for example, pre-emergence applications followed by post-emergence applications or using early post-emergence applications followed by medium or late post-emergence applications. Preference is given here to the joint or almost simultaneous application of the active compounds of the combination in question.

The herbicides belonging to any of the above defined groups (A), (B), (C) and (D) and to be applied according to present invention can be converted jointly or separately into customary formulations, such as solutions, emulsions suspensions, powders, foams, pastes, granules, aerosols, natural and synthetic materials impregnated with active compound and microencapsulations in polymeric materials. The formulations may comprise the customary auxiliaries and additives.

These formulations are produced in a known manner, for example by mixing the active compounds with extenders, that is liquid solvents, pressurized liquefied gases and/or solid carriers, if appropriate with the use of surfactants, that is emulsifiers and/or dispersants, and/or foam formers.

If the extender used is water, it is also possible to use, for example, organic solvents as auxiliary solvents. Suitable liquid solvents are essentially: aromatics, such as xylene, toluene, alkylnaphthalenes, chlorinated aromatics or chlorinated aliphatic hydrocarbons, such as chlorobenzenes, chloroethylenes, or methylene chloride, aliphatic hydrocarbons, such as cyclohexane or paraffins, for example mineral oil fractions, mineral and vegetable oils, alcohols, such as butanol or glycol, and ethers and esters thereof, ketones, such as acetone, methyl ethyl ketone, methyl isobutyl ketone or cyclohexanone, strongly polar solvents, such as dimethylformamide or dimethyl sulfoxide, and also water. Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, 30 alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as sawdust, coconut shells, corn cobs and tobacco stalks; suitable emulsifiers and/or foam formers are: for example nonionic and anionic emulsifiers, such as polyoxyethylene fatty acid esters, polyoxyethylene fatty alcohol 5 ethers, for example alkylaryl polyglycol ethers, alkylsulfonates, alkyl sulfates, arylsulfonates and also protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, and also natural phospholipids, such as cephalins and lecithins and synthetic phospholipids, can be used in the formulations. Other possible additives are mineral and vegetable oils.

The herbicidal action of the herbicide combinations to be used according to the invention can be improved, for example, by surfactants, preferably by wetting agents from the group of the fatty alcohol polyglycol ethers. The fatty alcohol polyglycol ethers preferably comprise 10-18 carbon atoms in the fatty alcohol radical and 2-20 ethylene oxide units in the polyglycol ether moiety. The fatty alcohol polyglycol ethers may be present in nonionic form, or ionic form, for example in the form of fatty alcohol polyglycol ether sulfates, which may be used, for example, as alkali metal salts (for example sodium salts and potassium salts) or ammonium salts, or even as alkaline earth metal salts, such as magnesium salts, such as C12/C14-fatty alcohol diglycol ether sulfate sodium (Genapol® LRO, Clariant GmbH); see, for example, EP-A-0476555, EP-A-0048436, EP-A-0336151 or U.S. Pat. No. 4,400,196 and also Proc. EWRS Symp. “Factors Affecting Herbicidal Activity and Selectivity”, 227-232 (1988). Nonionic fatty alcohol polyglycol ethers are, for example, (C10-C1a)-, preferably (C10-C14)-fatty alcohol polyglycol ethers (for example isotridecyl alcohol polyglycol ethers) which comprise, for example, 2-20, preferably 3-15, ethylene oxide units, for example those from the Genapol® X-series, such as Genapol® X-030, Genapol® X-060, Genapol® X-080 or Genapol® X-150 (all from Clariant GmbH).

The present invention further comprises the combination of ALS inhibitor herbicides belonging to any of the groups (A), (B), and (C) according to present invention with the wetting agents mentioned above from the group of the fatty alcohol polyglycol ethers which preferably contain 10-18 carbon atoms in the fatty alcohol radical and 2-20 ethylene oxide units in the polyglycol ether moiety and which may be present in nonionic or ionic form (for example as fatty alcohol polyglycol ether sulfates). Preference is given to C12/C14-fatty alcohol diglycol ether sulfate sodium (Genapol® LRO, Clariant GmbH) and isotridecyl alcohol polyglycol ether having 3-15 ethylene oxide units, for example from the Genapol® X-series, such as Genapol® X-030, Genapol® X-060, Genapol® X-080 and Genapol® X-150 (all from Clariant GmbH).

Furthermore, it is known that fatty alcohol polyglycol ethers, such as nonionic or ionic fatty alcohol polyglycol ethers (for example fatty alcohol polyglycol ether sulfates) are also suitable for use as penetrants and activity enhancers for a number of other herbicides (see, for example, EP-A-0502014).

The herbicidal action of the herbicide combinations according to the invention can also be enhanced by using vegetable oils. The term vegetable oils is to be understood as meaning oils of oleaginous plant species, such as soybean oil, rapeseed oil, corn oil, sunflower oil, cottonseed oil, linseed oil, coconut oil, palm oil, thistle oil or castor oil, in particular rapeseed oil, and also their transesterification products, for example alkyl esters, such as rapeseed oil methyl ester or rapeseed oil ethyl ester.

The vegetable oils are preferably esters of C10-C22, preferably C12-C20-, fatty acids. The C10-C22 fatty acid esters are, for example, esters of unsaturated or saturated C10-C22 fatty acids, in particular those having an even number of carbon atoms, for example erucic acid, lauric acid, palmitic acid and in particular C18-fatty acids, such as stearic acid, oleic acid, linoleic acid or linolenic acid.

Examples of C10-C22 fatty acid esters are esters obtained by reacting glycerol or glycol with the C10-C22 fatty acids contained, for example, in oils of oleaginous plant species, or C1-C20-alkyl-C10-C22 fatty acid esters which can be obtained, for example, by transesterification of the aforementioned glycerol- or glycol-C10-C22 fatty acid esters with C1-C20-alcohols (for example methanol, ethanol, propanol or butanol). The transesterification can be carried out by known methods as described, for example, in Rompp Chemie Lexikon, 9th edition, Volume 2, page 1343, Thieme Verlag Stuttgart.

Preferred C1-C20-alkyl-C10-C22 fatty acid esters are methyl esters, ethyl esters, propyl esters, butyl esters, 2-ethylhexyl esters and dodecyl esters. Preferred glycol- and glycerol-C10-C2rfatty acid esters are the uniform or mixed glycol esters and glycerol esters of C10-C2rfatty acids, in particular fatty acids having an even number of carbon atoms, for example erucic acid, lauric acid, palmitic acid and, in particular, C18-fatty acids, such as stearic acid, oleic acid, linoleic acid or linolenic acid.

In the herbicidal compositions to be used according to the invention, the vegetable oils can be present, for example, in the form of commercially available oil-containing formulation additives, in particular those based on rapeseed oil, such as Hasten® (Victorian Chemical Company, Australia, hereinbelow referred to as Hasten, main ingredient: rapeseed oil ethyl ester), Actirob®B (Novance, France, hereinbelow referred to as ActirobB, main ingredient: rapeseed oil methyl ester), Rako-Binol® (Bayer AG, Germany, hereinbelow referred to as Rako-Binol, main ingredient:rapeseed oil), Renal® (Stefes, Germany, hereinbelow referred to as Renal, vegetable oil ingredient: rapeseed oil methyl ester) or Stefes Mero® (Stefes, Germany, hereinbelow referred to as Mero, main ingredient: rapeseed oil methyl ester).

It is possible to use colorants, such as inorganic pigments, for example iron oxide, titanium oxide, Prussian Blue, and organic dyes, such as alizarin dyes, azo dyes and metal phthalocyanine dyes, and trace nutrients such as salts of iron, manganese, boron, copper, cobalt, molybdenum and zinc.

The formulations to be used according to present invention generally comprise from 0.1 to 95% by weight of active compounds, preferably from 0.5 to 90% by weight.

As such or in their formulations, the ALS inhibitor herbicides belonging to any of the above defined groups (A), (B), and (C) can also be used as a mixture with other agrochemical active compounds, such as known non-ALS inhibitor herbicides, for controlling unwanted vegetation, for example for controlling weeds or for controlling unwanted crop plants, finished formulations or tank mixes, for example, being possible.

The use of a mixture of ALS inhibitor herbicides belonging to any of the above 30 defined groups (A), (B), and (C) with other known active compounds, such as fungicides, insecticides, acaricides, nematicides, safeners, bird repellants, plant nutrients and soil structure improvers is likewise possible.

The ALS inhibitor herbicides belonging to any of the above defined groups (A), (B), (C) can be used as such, in the form of their formulations or in the use forms prepared therefrom by further dilution, such as ready-to-use solutions, suspensions, emulsions, powders, pastes and granules. Application is carried out in a customary manner, for example by watering, spraying, atomizing, broadcasting.

According to the invention, one or more of the ALS inhibitor herbicides belonging to any of the above defined groups (A), (B), and (C) can be applied either alone or in combination with one or more non-ALS inhibitor herbicides belonging to group (D) to the plants (for example harmful plants, such as monocotyledonous or dicotyledonous weeds or unwanted crop plants), the seed (for example grains, seeds or vegetative propagation organs, such as tubers or shoot parts with buds) or the area under cultivation (for example the soil), preferably to the green plants and parts of plants and, if appropriate, additionally the soil. One possible use is the joint application of the active compounds in the form of tank mixes, where the optimally formulated concentrated formulations of the individual active compounds are, together, mixed in a tank with water, and the spray liquor obtained is applied.

### Additional Definitions

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “plant” includes plant cells, plant protoplasts, plant cells of tissue culture from which beet plants can be regenerated, plant calli, plant clumps and plant cells that are intact in plants or parts of plants such as pollen, flowers, seeds, leaves, stems, and the like. Also included is propagation material and harvestable parts such as roots, particularly beet roots.

Parts of plants may be attached to or separate from a whole intact plant. Such parts of a plant include, but are not limited to, organs, tissues, and cells of a plant, and preferably seeds.

As used herein, the term “population” means a genetically heterogeneous collection of plants that share a common parental derivation.

As used herein, the terms “variety” and “cultivar” mean a group of similar plants that by their genetic pedigrees and performance can be identified from other varieties within the same species.

As used herein, an “allele” refers to one of two or more alternative forms of a genomic sequence at a given locus on a chromosome.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of a plant.

As used herein, “elite” or “cultivated” variety or line means any variety that has resulted from breeding and selection for superior agronomic performance. An “elite plant” refers to a plant belonging to an elite variety or line. Numerous elite varieties are available and known to those of skill in the art of beet breeding. An “elite population” is an assortment of elite individuals or varieties that can be used to represent the state of the art in terms of agronomically superior genotypes of a given crop species, such as beet. Similarly, an “elite germplasm” or elite strain of germplasm is an agronomically superior germplasm.

As used herein, the term “introgressed,” when used in reference to a genetic locus, refers to a genetic locus that has been introduced into a new genetic background, such as through backcrossing. Introgression of a genetic locus can be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic loci in a configuration in which they are not found in nature, for example as a result of a recombination event between homologous chromosomes during meiosis.

As used herein, the term “linked,” when used in the context of nucleic acid markers and/or genomic regions, means that the markers and/or genomic regions are located on the same linkage group or chromosome such that they tend to segregate together at meiosis.

“Sequence identity” and “sequence similarity” can be determined by alignment of r two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters) share at least a certain minimal percentage of sequence identity. These programs use the Needleman and Wunsch global alignment algorithm to align two sequences over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL (Henikoff & Henikoff, 1992, PNAS 89, 10915-10919). Sequence alignments and scores for percentage sequence identity may for example be determined using computer programs, such as EMBOSS, as available on the world wide web under ebi.ac.uk/Tools/psa/emboss_needle/. Alternatively, sequence similarity or identity may be determined by searching against databases such as FASTA, BLAST, etc., but hits should be retrieved and aligned pairwise to compare sequence identity. Two nucleic acid sequences have “substantial sequence identity” if the percentage sequence identity is at least 85%, 90%, 95%, 98%, 99% or more (e.g. at least 99.1, 99.2 99.3 99.4, 99.5, 99.6, 99.7, 99.8, 99.9 or more (as determined by Emboss “needle” using default parameters, i.e. gap creation penalty=10, gap extension penalty=0.5, using scoring matrix DNAFULL for nucleic acids). Markers may sometimes exhibit variation, particularly in regions which are not recognized by the probes.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

An “endogenous” gene means a gene of a plant which has not been introduced into the plant by genetic engineering techniques.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

All publications and patents cited in this disclosure are incorporated by reference in their entirety. To the extent the material incorporated by reference contradicts or is inconsistent with this specification, the specification will supersede any such material.

### DEPOSIT INFORMATION

Seeds of the ALS inhibitor tolerant Beta vulgaris donor line comprising the BVals_W569L allele and referred herein as SU-12-1 have been deposited with the NCIMB, Aberdeen, UK, under Number NCIMB 41705 on Mar. 12, 2010.

Throughout the description, reference is made to the following Sequence Listing entries:
SEQ ID NO. 1: amino acid sequence of a large ALS subunit from wild type reference Beta vulgaris.
SEQ ID NO. 2: nucleotide sequence encoding large ALS subunit from wild type reference Beta vulgaris.
SEQ ID NO. 3: amino acid sequence of a large herbicide tolerant ALS subunit from Beta vulgaris (BvALS_W569L)
SEQ ID NO. 4: nucleotide sequence encoding a large herbicide tolerant ALS subunit from Beta vulgaris (BvALS_W569L)
SEQ ID NO. 5: amino acid sequence of a small regulatory ALS subunit from Arabidopsis thaliana (At2g31810)
SEQ ID NO. 6: nucleotide sequence of a small regulatory ALS subunit from Arabidopsis thaliana (At2g31810)
SEQ ID NO. 7: amino acid sequence of a small regulatory ALS subunit from Arabidopsis thaliana (At5g16290)
SEQ ID NO. 8: nucleotide sequence of a small regulatory ALS subunit from Arabidopsis thaliana (At5g16290)
SEQ ID NO. 9: amino acid sequence of a small regulatory ALS subunit from reference Beta vulgaris (B3_059040)
SEQ ID NO. 10: nucleotide sequence of a small regulatory ALS subunit from reference Beta vulgaris (B3_059040)
SEQ ID NO. 11: amino acid sequence of a small regulatory ALS subunit from reference Beta vulgaris (B4_074570)
SEQ ID NO. 12: nucleotide sequence of a small regulatory ALS subunit from reference Beta vulgaris (B4_074570)
SEQ ID NO. 13: amino acid sequence of a small regulatory ALS subunit from Beta vulgaris Genotype A (B3_059040)
SEQ ID NO. 14: nucleotide sequence of a small regulatory ALS subunit from Beta vulgaris Genotype A (B3_059040)
SEQ ID NO. 15: amino acid sequence of a small regulatory ALS subunit from Beta vulgaris Genotype A (B4_074570)
SEQ ID NO. 16: nucleotide sequence of a small regulatory ALS subunit from Beta vulgaris Genotype A (B4_074570)
SEQ ID NO. 17: amino acid sequence of a small regulatory ALS subunit from Beta vulgaris Genotype B (B3_059040)
SEQ ID NO. 18: nucleotide sequence of a small regulatory ALS subunit from Beta vulgaris Genotype B (B3_059040)
SEQ ID NO. 19: amino acid sequence of a small regulatory ALS subunit from Beta vulgaris Genotype B (B4_074570)
SEQ ID NO. 20: nucleotide sequence of a small regulatory ALS subunit from Beta vulgaris Genotype B (B4_074570)
SEQ ID NO. 21: amino acid sequence of a small regulatory ALS subunit from Beta vulgaris Genotype C (B3_059040)
SEQ ID NO. 22: nucleotide sequence of a small regulatory ALS subunit from Beta vulgaris Genotype C (B3_059040)
SEQ ID NO. 23: amino acid sequence of a small regulatory ALS subunit from Beta vulgaris Genotype C (B4_074570)
SEQ ID NO. 24: nucleotide sequence of a small regulatory ALS subunit from Beta vulgaris Genotype C (B4_074570)
SEQ ID NO. 25: amino acid sequence of a small regulatory ALS subunit from Beta vulgaris Genotype D (B3_059040)
SEQ ID NO. 26: nucleotide sequence of a small regulatory ALS subunit from Beta vulgaris Genotype D (B3_059040)
SEQ ID NO. 27: amino acid sequence of a small regulatory ALS subunit from Beta vulgaris Genotype D (B4_074570)
SEQ ID NO. 28: nucleotide sequence of a small regulatory ALS subunit from Beta vulgaris Genotype D (B4_074570)
SEQ ID NO. 29: amino acid sequence of a small regulatory ALS subunit from Beta vulgaris Genotype E (B3_059040)
SEQ ID NO. 30: nucleotide sequence of a small regulatory ALS subunit from Beta vulgaris Genotype E (B3_059040)
SEQ ID NO. 31: amino acid sequence of a small regulatory ALS subunit from Beta vulgaris Genotype E (B4_074570)
SEQ ID NO. 32: nucleotide sequence of a small regulatory ALS subunit from Beta vulgaris Genotype E (B4_074570)
SEQ ID NO. 33: nucleotide sequence of marker M1 for the identification of a small regulatory ALS subunit from Beta vulgaris (B3_059040)
SEQ ID NO. 34: nucleotide sequence of marker M2 for the identification of a small regulatory ALS subunit from Beta vulgaris (B3_059040)
SEQ ID NO. 35: nucleotide sequence of marker M3 for the identification of a small regulatory ALS subunit from Beta vulgaris (B3_059040)
SEQ ID NO. 36: nucleotide sequence of marker M4 for the identification of a small regulatory ALS subunit from Beta vulgaris (B4_074570)
SEQ ID NO. 37: nucleotide sequence of marker M5—left marker for a small regulatory ALS subunit from Beta vulgaris (B3_059040)
SEQ ID NO. 38: nucleotide sequence of marker M6—left marker for a small regulatory ALS subunit from Beta vulgaris (B3_059040)
SEQ ID NO. 39: nucleotide sequence of marker M7—left marker for a small regulatory ALS subunit from Beta vulgaris (B3_059040)
SEQ ID NO. 40: nucleotide sequence of marker M8—left marker for a small regulatory ALS subunit from Beta vulgaris (B4_074570)
SEQ ID NO. 41: nucleotide sequence of marker M9—left marker for a small regulatory ALS subunit from Beta vulgaris (B4_074570)
SEQ ID NO. 42: nucleotide sequence of marker M10—left marker for a small regulatory ALS subunit from Beta vulgaris (B4_074570)
SEQ ID NO. 43: nucleotide sequence of marker M11—right marker for a small regulatory ALS subunit from Beta vulgaris (B3_059040)
SEQ ID NO. 44: nucleotide sequence of marker M12—right marker for a small regulatory ALS subunit from Beta vulgaris (B3_059040)
SEQ ID NO. 45: nucleotide sequence of marker M13—right marker for a small regulatory ALS subunit from Beta vulgaris (B3_059040)
SEQ ID NO. 46: nucleotide sequence of marker M14—right marker for a small regulatory ALS subunit from Beta vulgaris (B4_074570)
SEQ ID NO. 47: nucleotide sequence of marker M15—right marker for a small regulatory ALS subunit from Beta vulgaris (B4_074570)
SEQ ID NO. 48: nucleotide sequence of marker M16—right marker for a small regulatory ALS subunit from Beta vulgaris (B4_074570)

### Examples

### Impact of Different Regulatory Subunit Variants on the Activity of the BvALS Holoenzyme

### Material & Methods

The coding sequences of the catalytic BvALS subunit and the regulatory BvALS subunits are PCR-amplified from sugar beet cDNA and cloned into vectors suitable for protein expression. Concerning the catalytical subunit also the herbicide-tolerant variants W569L are included.

The proteins are either expressed in a bacterial system using the pET-28 vector (Novagen/Merck) or in a system based on plant cells using the pALiCE vectors (LenioBio). Protein purification is done via a His-tag. The determination of the enzyme activity is either done using a fixed colorimetric assay measuring acetoin converted from acetolactate or using a continuous assay monitoring the consumption of pyruvate (Chang et al., 1997 Expression, purification and characterization of Arabidopsis thaliana acetohydroxyacid synthase).

### Results and Discussion

ALS activity assays performed using the catalytic subunit only, result in different values compared to assays based on in vitro reconstituted holoenzymes. Combining the regulatory subunits with the purified catalytic subunit results in an activity stimulation that is sensitive to inhibition by valine, leucine, and isoleucine as demonstrated in Arabidopsis thaliana (Lee et al., 2001).

The enzyme activity of the ALS holoenzyme without herbicide-tolerance mutation is measured in comparison to herbicide-tolerant ALS holoenzymes possessing different combinations of regulatory subunits reflecting and testing the allelic variation found in the panel of relevant sugar beet genotypes. This approach identifies the combinations of catalytical and regulatory ALS subunits performing best to ensure robust enzyme performance.

### Identification of BvALS Regulatory Subunits

Identification of Beta vulgaris orthologues is based on the published Arabidopsis thaliana genes coding for the ALS regulatory subunits. At2g31810 (SEQ ID Nos. 5 and 6) is a validated Arabidopsis ALS regulatory subunit while a close homologue At5g16290 (SEQ ID Nos. 7 and 8) is classified as potential ALS regulatory subunit. For both genes the sugar beet orthologues are found by BLAST. BV3_059040 corresponds to At2g31810 and BV4_074570 corresponds to At5g16290. The coding sequences extracted from reference genotype and predicted amino acid sequences can be found in SEQ ID Nos 9 to 12 of the sequence listing.

Based on sequence analyses of best performing genotypes, the best fitting combinations of regulatory subunits and mutant catalytical subunit (W569L) conferring ALS inhibitor herbicide tolerance ensuring robust BvALS enzyme performance are referred to as genotypes A, B, C, D and E. Their respective amino acid sequences and nucleotide sequences for both BV3_059040 and BV4_074570 can be found in the sequence listing (Seq ID NOs 13-32). See also paragraph [00114].

Marker suitable to follow Bv3_059040 and Bv4_074570 alleles during breeding process and product development are listed in the tables below and in the sequence listing.

### Markers on the Gene(s):

| | SBKv7* | | | Markers located in the gene | | | |
|---|---|---|---|---|---|---|---|
| | | Start | End | Marker | | | Marker |
| Gene | Chromosome | [bp] | [bp] | name | Assay | Position | sequence |
| Bv3-059040 | 3 | 17473613 | 17484979 | M1 | INF-XT28k | 3: 17483735 . . . 17483785 | SEQ ID NO: 33 |
| | | | | M2 | INF-XT28k | 3: 17478960 . . . 17479010 | SEQ ID NO: 34 |
| | | | | M3 | INF-XT28k | 3: 17476433 . . . 17476483 | SEQ ID NO: 35 |
| Bv4_074570 | 4 | 3601108 | 360986 | M4 | KSP | 4: 3601440 . . . 3601490 | SEQ ID NO: 36 |

*reference Beta vulgaris genotype (sequences see above)

### Left Flanking Markers:

| | SBKv7* | | | Markers located left flanking | | | |
|---|---|---|---|---|---|---|---|
| | | Start | End | Marker | | | Marker |
| Gene | Chromosome | [bp] | [bp] | name | Assay | Position | sequence |
| Bv3-059040 | 3 | 17473613 | 17484979 | M5 | INF-XT28k | 3: 17447436 . . . 17447486 | SEQ ID NO: 37 |
| | | | | M6 | INF-XT28k | 3: 17445956 . . . 17446006 | SEQ ID NO: 38 |
| | | | | M7 | INF-XT28k | 3: 17440542 . . . 17440592 | SEQ ID NO: 39 |
| Bv4_074570 | 4 | 3601108 | 360986 | M8 | INF-XT28k | 4: 3598685 . . . 3598735 | SEQ ID NO: 40 |
| | | | | M9 | INF-XT28k | 4: 3594009 . . . 3594059 | SEQ ID NO: 41 |
| | | | | M10 | INF-XT28k | 4: 3579094 . . . 3579144 | SEQ ID NO: 42 |

### Right Flanking Markers:

| | SBKv7* | | | Markers located right flanking | | | |
|---|---|---|---|---|---|---|---|
| | | Start | End | Marker | | | Marker |
| Gene | Chromosome | [bp] | [bp] | name | Assay | Position | sequence |
| Bv3-059040 | 3 | 17473613 | 17484979 | M11 | INF-XT28k | 3: 17519446 . . . 17519496 | SEQ ID NO: 43 |
| | | | | M12 | INF-XT28k | 3: 17600615 . . . 17600665 | SEQ ID NO: 44 |
| | | | | M13 | INF-XT28k | 3: 17668482 . . . 17668532 | SEQ ID NO: 45 |
| Bv4_074570 | 4 | 3601108 | 360986 | M14 | INF-XT28k | 4: 3623798 . . . 3623848 | SEQ ID NO: 46 |
| | | | | M15 | INF-XT28k | 4: 3646135 . . . 3646185 | SEQ ID NO: 47 |
| | | | | M16 | INF-XT28k | 4: 3648995 . . . 3649045 | SEQ ID NO: 48 |

*reference Beta vulgaris genotype (sequences see above)

1. An acetolactate synthase (ALS) inhibitor-herbicide tolerant Beta vulgaris plant or seed, such as a sugar beet plant or seed, comprising an ALS holo-enzyme comprising
a. a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan; and
b. a small subunit of ALS which can be selected by identification with marker M1 (comprising the nucleotide sequence of SEQ ID NO. 33), marker M2 (comprising the nucleotide sequence of SEQ ID NO. 34), marker M3 (comprising the nucleotide sequence of SEQ ID NO. 35) or marker M4 (comprising the nucleotide sequence of SEQ ID NO. 36).

2. The Beta vulgaris plant or seed of claim 1, wherein said small subunit of ALS is encoded by a chromosomal region located on chromosome 3, between a marker selected from marker M5 (comprising the nucleotide sequence of SEQ ID NO. 37), marker M6 (comprising the nucleotide sequence of SEQ ID NO. 38) or marker M7 (comprising the nucleotide sequence of SEQ ID NO. 39) and a marker selected from marker M11 (comprising the nucleotide sequence of SEQ ID NO. 43), marker M12 (comprising the nucleotide sequence of SEQ ID NO. 44) or marker M13 (comprising the nucleotide sequence of SEQ ID NO. 45) or wherein the small subunit of ALS is encoded by a chromosomal region located on chromosome 4, between a marker selected from marker M8 (comprising the nucleotide sequence of SEQ ID NO. 40), marker M9 (comprising the nucleotide sequence of SEQ ID NO. 41) or marker M10 (comprising the nucleotide sequence of SEQ ID NO. 42) and a marker selected from marker M14 (comprising the nucleotide sequence of SEQ ID NO. 46), marker M15 (comprising the nucleotide sequence of SEQ ID NO. 47) or marker M16 (comprising the nucleotide sequence of SEQ ID NO. 48).

3. The Beta vulgaris plant or seed of claim 1, wherein said small subunit of ALS comprises an amino acid sequence having at least 95%, or at least 98% sequence identity with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31 or is encoded by a nucleotide sequence having at least 95% sequence identity with a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.

4. The Beta vulgaris plant or seed of claim 1, comprising:
a. an allele of a small subunit of ALS on chromosome 3 encoding an amino acid sequence having 98% sequence identity to the amino acid sequence of SEQ ID NO. 13 and further comprising an allele of a small subunit of ALS on chromosome 4 encoding an amino acid sequence having 98% sequence identity to the amino acid sequence of SEQ ID NO. 15;
b. an allele of a small subunit of ALS on chromosome 3 encoding an amino acid sequence having 98% sequence identity to the amino acid sequence of SEQ ID NO. 17 and further comprising an allele of a small subunit of ALS on chromosome 4 encoding an amino acid sequence having 98% sequence identity to the amino acid sequence of SEQ ID NO. 19;
c. an allele of a small subunit of ALS on chromosome 3 encoding an amino acid sequence having 98% sequence identity to the amino acid sequence of SEQ ID NO. 21 and further comprising an allele of a small subunit of ALS on chromosome 4 encoding an amino acid sequence having 98% sequence identity to the amino acid sequence of SEQ ID NO. 23;
d. an allele of a small subunit of ALS on chromosome 3 encoding an amino acid sequence having 98% sequence identity to the amino acid sequence of SEQ ID NO. 25 and further comprising an allele of a small subunit of ALS on chromosome 4 encoding an amino acid sequence having 98% sequence identity to the amino acid sequence of SEQ ID NO. 27; or
e. an allele of a small subunit of ALS on chromosome 3 encoding an amino acid sequence having 98% sequence identity to the amino acid sequence of SEQ ID NO. 29 and further comprising an allele of a small subunit of ALS on chromosome 4 encoding an amino acid sequence having 98% sequence identity to the amino acid sequence of SEQ ID NO. 31;
or comprising:
f. an allele of a small subunit of ALS on chromosome 3 comprising a nucleotide sequence having 98% sequence identity to the nucleotide sequence of SEQ ID NO. 14 and further comprising an allele of a small subunit of ALS on chromosome 4 comprising a nucleotide sequence having 98% sequence identity to the nucleotide sequence of SEQ ID NO. 16;
g. an allele of a small subunit of ALS on chromosome 3 comprising a nucleotide sequence having 98% sequence identity to the nucleotide sequence of SEQ ID NO. 18 and further comprising an allele of a small subunit of ALS on chromosome 4 comprising a nucleotide sequence having 98% sequence identity to the nucleotide sequence of SEQ ID NO. 20;
h. an allele of a small subunit of ALS on chromosome 3 comprising a nucleotide sequence having 98% sequence identity to the nucleotide sequence of SEQ ID NO. 22 and further comprising an allele of a small subunit of ALS on chromosome 4 comprising a nucleotide sequence having 98% sequence identity to the nucleotide sequence of SEQ ID NO. 24;
i. an allele of a small subunit of ALS on chromosome 3 comprising a nucleotide sequence having 98% sequence identity to the nucleotide sequence of SEQ ID NO. 26 and further comprising an allele of a small subunit of ALS on chromosome 4 comprising a nucleotide sequence having 98% sequence identity to the nucleotide sequence of SEQ ID NO. 28; or
j. an allele of a small subunit of ALS on chromosome 3 comprising a nucleotide sequence having 98% sequence identity to the nucleotide sequence of SEQ ID NO. 30 and further comprising an allele of a small subunit of ALS on chromosome 4 comprising a nucleotide sequence having 98% sequence identity to the nucleotide sequence of SEQ ID NO. 32.

5. The Beta vulgaris plant or seed of claim 1, wherein said large subunit of ALS comprises the amino acid sequence of SEQ ID NO. 3 or wherein said large subunit of ALS is encoded by a nucleotide sequence comprises the nucleotide sequence of SEQ ID NO. 4.

6. A method for producing a Beta vulgaris plant with optimally fitted large subunit and one or more regulatory subunits of an ALS holoenzyme comprising:
a. crossing a Beta vulgaris plant comprising an allele encoding a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan, with a Beta vulgaris plant comprising at least one allele encoding a small subunit of ALS comprises an amino acid sequence having at least 95% sequence identity with an amino acid sequence selected from the group of SEQ ID NO. 13, SEQ ID NO. 15, SEQ ID NO. 17, SEQ ID NO. 19; SEQ ID NO 21; SEQ ID NO. 23, SEQ ID NO; 25; SEQ ID NO. 27; SEQ ID NO. 29 or SEQ ID NO. 31; and
b. identifying progeny plants comprising said allele encoding said large subunit of ALS and said at least one allele encoding said regulatory subunit of ALS.

7. A method for producing a Beta vulgaris plant with optimally fitted large subunit and one or more regulatory subunits of an ALS holoenzyme comprising:
a. providing a Beta vulgaris plant comprising an allele encoding a large subunit of ALS comprising an amino acid sequence having at least 95% sequence identity with the amino acid sequence of SEQ ID NO. 1 and further comprising a leucine at a position corresponding to amino acid position 569 instead of the naturally occurring tryptophan, such as an amino acid sequence of SEQ ID NO. 3;
b. adapt, by genome-editing or directed mutation, the nucleotide sequence of the allele on chromosome 3 and/or the allele on chromosome 4 encoding a small subunit of ALS to obtain a nucleotide sequence selected from the group of SEQ ID NO. 14, SEQ ID NO. 16, SEQ ID NO. 18, SEQ ID NO. 20; SEQ ID NO 22; SEQ ID NO. 24, SEQ ID NO; 26; SEQ ID NO. 28; SEQ ID NO. 30 or SEQ ID NO. 32.

8. Use of a hybrid Beta vulgaris plant according to claim 1 for the production of sugar, ethanol, betaine and/or uridine of for the production of animal feed.

9. Use of one or more ALS inhibitor herbicide(s) for controlling unwanted vegetation in Beta vulgaris growing areas wherein the Beta vulgaris plants are hybrid Beta vulgaris plants according to claim 1.

10. Use of one or more ALS inhibitor herbicide(s) according to claim 9, wherein the ALS inhibitor herbicide(s) comprises foramsulfuron [CAS RN 173159-57-4] (=A1-13) and thiencarbazone-methyl [CAS RN 317815-83-1] (=A2-3) or iodosulfuron-methyl-sodium [CAS RN 144550-36-7] (=A1-16) and thiencarbazone-methyl [CAS RN 317815-83-1] (=A2-3).

11. Use of one or more ALS inhibitor herbicide(s) according to claim 9 in combination with non-ALS inhibitor herbicides (i.e. herbicides showing a mode of action that is different to the inhibition of the ALS enzyme [acetohydroxyacid synthase; EC 2.2.1.6] group D herbicides), and wherein the non-ALS inhibitor herbicide(s) is/are selected form the group consisting of chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, and sethoxydim.

12. Method for controlling unwanted vegetation in Beta vulgaris plant growing areas, characterized by:
(a) the presence of Beta vulgaris plants according to claim 1;
(b) the application of one or more ALS inhibitor herbicide(s) alone or in combination with one or more herbicide(s) that do(es) not belong to the class of ALS inhibitor herbicides (non-ALS inhibitor herbicides), and
(c) wherein the application of the respective herbicides as defined under (b)
(i) takes place jointly or simultaneously, or
(ii) takes place at different times and/or in a plurality of portions (sequential application), in pre-emergence applications followed by post-emergence applications or early post-emergence applications followed by medium or late post-emergence applications.

13. Method according to claim 12 for controlling unwanted vegetation, wherein the ALS inhibitor herbicide(s) comprise foramsulfuron [CAS RN 173159-57-4] (=A1-13) and thiencarbazone-methyl [CAS RN 317815-83-1] (=A2-3) or iodosulfuron-methyl-sodium [CAS RN 144550-36-7] (=A1-16) and thiencarbazone-methyl [CAS RN 317815-83-1] (=A2-3).

14. Method according to claim 12, wherein the non-ALS inhibitor herbicide(s) are taken from the group consisting of chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl and sethoxydim.


SEQUENCE LISTING (48 sequences)

SEQ ID NO: 1 - protein, 660 aa, Beta vulgaris
MAATFTNPTFSPSSTPLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSKWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSKSADIFPDMLKFAEACDIPSARVSNVADLRAAIQTMLDTPGPYLLDVIVPHQEHVLPMIPSGAGFKDTITEGD

SEQ ID NO: 2 - DNA, 1998 bp, Beta vulgaris
  source 1..1998: mol_type = genomic DNA
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 3 - protein, 660 aa, Beta vulgaris
MAATFTNPTFSPSSTPLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSKWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQLEDRFYKANRAHTYLGNPSKSADIFPDMLKFAEACDIPSARVSNVADLRAAIQTMLDTPGPYLLDVIVPHQEHVLPMIPSGAGFKDTITEGD

SEQ ID NO: 4 - DNA, 1998 bp, Beta vulgaris
  source 1..1998: mol_type = genomic DNA
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaattggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 5 - protein, 491 aa, Arabidopsis thaliana
MAAISVSSSPSIRCLRSACSDSSPALVSSTRVSFPAKISYLSGISSHRGDEMGKRMEGFVRSVDGKISDASFSEASSATPKSKVRKHTISVFVGDESGMINRIAGVFARRGYNIESLAVGLNRDKALFTIVVCGTERVLQQVIEQLQKLVNVLKVEDISSEPQVERELMLVKVNAHPESRAEIMWLVDTFRARVVDIAEHALTIEVTGDPGKMIAVERNLKKFQIREIVRTGKIALRREKMGATAPFWRFSAASYPDLKEQAPVSVLRSSKKGAIVPQKETSAGGDVYPVEPFFDPKVHRILDAHWGLLTDEDTSGLRSHTLSLLVNDIPGVLNIVTGVFARRGYNIQSLAVGHAETKGISRITTVIPATDESVSKLVQQLYKLVDVHEVHDLTHLPFSERELMLIKIAVNAAARRDVLDIASIFRAKAVDVSDHTITLQLTGDLDKMVALQRLLEPYGICEVARTGRVALARESGVDSKYLRGYSFPLTG

SEQ ID NO: 6 - DNA, 1675 bp, Arabidopsis thaliana
  source 1..1675: mol_type = genomic DNA
cgtcgtcagtcttcttcagtagcaaaaaaccttcggcttcgtctcgtcaatggcggccatttctgtaagttcttcaccatctattcgctgcttgagatcggcatgttccgattcttctcctgctcttgtatcctcgacgcgtgtatcgttcccggcgaagatttcatatctctccggtatatcttcgcaccgtggcgatgaaatgggtaagagaatggaaggattcgttagaagcgtcgatgggaagatctctgatgcgtctttctccgaagcttcatctgcgactccaaaatcgaaggtgaggaagcacacaatttcagtatttgttggagacgaaagcggaatgattaataggattgcaggagtgtttgcaaggagaggatacaatattgagagtcttgctgttggtctgaacagagacaaggctctattcaccatagttgtctgtggaactgaaagggtacttcagcaggtcatcgagcaactccagaagctcgttaatgttctaaaggttgaagatatctcaagtgagccgcaagtggagcgtgagctgatgcttgtaaaagtgaatgcacatccagaatccagggcagagatcatgtggctagttgacacattcagagcaagagttgtagatatagcggaacatgcattgactatcgaggtaactggagatcctggaaaaatgattgctgtagaaagaaatttgaaaaagtttcagatcagagagattgtaaggacaggaaagatagcactgagaagggaaaagatgggtgcaactgctccattttggcgattttcagcagcatcctatccagatctcaaggagcaagcgcctgttagtgttcttcgaagtagcaaaaaaggagccattgtccctcaaaaggaaacatcagcagggggagatgtttatcccgttgagccattttttgaccccaaggtacatcgtattctcgacgctcactggggacttctcactgacgaagatacgagtggactacggtcgcatactctatcattgcttgtaaatgatattccaggagttcttaatattgtgactggtgttttcgctcgaaggggatacaatatccagagcttggccgtaggacatgctgaaaccaagggcatttcacgcattacaacagttatacctgcaacagatgaatcggtcagcaaattggtgcagcaactttacaaactcgtagatgtgcatgaggtccatgatcttactcatttgccattttctgaaagagaactgatgctgattaagattgccgtgaacgctgctgctagaagagatgtcctggacattgctagtattttcagggctaaagctgttgacgtatctgatcacacaattactttgcagcttactggggatctagacaagatggttgcactgcaaaggttattggagccctatggtatatgtgaggttgcaagaaccggtcgtgtggcattggctcgtgaatcgggagtggactccaagtaccttcgtggatactcctttcctttaacaggctaaaccgttgcagagtgcatccatcgaacatcagaaacttttgaaggtaaaagtttcattacacagtctatgaaccaactcaaagacagacagagagactgcgtagatatatgtttgtgactttgtttatgaaacaattagctgattttggcc

SEQ ID NO: 7 - protein, 477 aa, Arabidopsis thaliana
MAATTTATSLFSSRLHFQNQNQGYGFPAKTPNSLQVNQIIDGRKMRNATVLSAASTDKAITTAQSVAPTACDRVRRHTISVFVGDESGIINRIAGVFARRGYNIESLAVGLNEDKALFTIVVLGTDKVLQQVVEQLNKLVNVIKVEDLSKEPHVERELMLIKLNADPSTRSEIMWLVDIFRAKIVDTSEQSLTIEVTGDPGKMVALTTNLEKFGIKEIARTGKIALRREKMGETAPFWRFSAASYPHLVKESSHETVAEKTKLALTGNGNASSGGDVYPVEPYNDFKPVLDAHWGMVYDEDSSGLRSHTLSLLVANVPGVLNLITGAISRRGYNIQSLAVGPAEKEGLSRITTVIPGTDENIDKLVRQLQKLIDLQEIQNITHMPFAERELMLIKVAADTSARRDVLDIAQVFRAKAIDVSDHTITLEVTGDLRKMSALQTQLEAYGICEVARTGRVALVRESGVDSTYLRGYSLPL

SEQ ID NO: 8 - DNA, 1568 bp, Arabidopsis thaliana
  source 1..1568: mol_type = genomic DNA
tactctcccagaaatggcggcgacgacgactgctacttcattattctcatctcgtcttcacttccagaatcagaatcaaggttatgggtttccggcgaaaacccccaactccctgcaagtaaatcagatcatcgacgggaggaagatgaggaatgccactgttctctcagctgcttccactgataaagccatcaccactgctcaatctgtagctccgacggcttgtgatagagtaaggaggcatacaatctcggtgtttgtgggcgatgagagcggtataataaatagaattgctggagtatttgctagaaggggatacaatattgagtcccttgccgtaggattgaatgaagacaaggctctgtttactatagttgttcttggaacagataaagtcttgcagcaagttgtagaacagcttaacaagctcgtcaatgtcatcaaggtggaagatctctccaaggagccccatgtagaacgtgagttaatgctcattaaacttaacgctgatccaagcacccgctcagagatcatgtggttggtggatatctttagagcaaaaatcgtggatacctcagagcaatctctaacgattgaggtaactggagaccctggaaaaatggttgcactaacgacaaacttagagaagtttggaataaaagaaattgcgagaacagggaagattgctctgagacgggaaaagatgggagaaaccgctccgttttggagattttcagcggcatcatatccacatcttgtaaaggaatcatctcatgagactgttgcagagaaaacaaaactagcattgactggaaatggcaatgcatcatccgggggtgacgtttatccagtggaaccctataacgatttcaaaccggtccttgacgctcattggggaatggtctacgatgaagattcgagcggacttcggtcccacacattatctttacttgtggccaacgttcccggagttctcaatctaataactggagctatctctagaagaggttataacatccagagtctagctgttggtcctgctgagaaagagggcttatctcggattacaacagttattcccggaactgatgaaaatatagataaattggttaggcagctccagaagttgattgatcttcaggagatccaaaatataacgcacatgccatttgcggagcgagaactgatgctcatcaaagtagctgctgatacttctgcaaggagagatgttttagatattgctcaagttttccgtgctaaagccattgatgtctctgatcatactatcacccttgaggttacaggggatctcagaaaaatgtctgcattacaaacgcagttagaggcctatggaatctgcgaggtggctcggacaggaagagtggcattggtaagagaatctggagtggattcgacttatcttcgtggatactctcttcctttgtagttctagagaggacttgccgcaagcttcagtgtcgttcctggaaactagtcaaaaggactgcacgttgtttcagcatctgtgattgattgcttcctcacgagtcatggcgcttcgagttgtt

SEQ ID NO: 9 - protein, 486 aa, Beta vulgaris
MAAVSTHPSTSLNSCLKNEQTQKFIKSHAFLLKPQMLGFNPRKWKNLEFDKLVVSASNVDQQGNESNLPFNGVSSSTRSKAMRHTISVFVGDESGMINRIAGVFARRGYNIESLAVGLNEDKALFTIVVSGTDKVLHQVMEQLQKLLNVLKVEDISREPQVERELMLVKVGADQSSRGELMWLVDIFRAKIVDISEEYLTVEVTGDPGKMVAVLRNLSKFGIREIARTGKIALRREKLGESAPFWRFSAASYPDLKEATLVDALSEVARQAPDTGSSDFSVEGDVYPVEPYDGFAVPQVLDAHWGVLNDDTSGFQSHTLSMLVNDRPGVLNFVTGAFARRGYNIQSLAVGHAETKGLSRITTVVTGTDESISKLVQQIYKLVDIHEVKDLTHLPFAERELMLVKVAVNTAARREVLDIASIFRAKAVDVSDHTITLELTGDVNKMVALQRLLEPYGICEVARTGRVALARESGVDSRYLRGYSFPV

SEQ ID NO: 10 - DNA, 1461 bp, Beta vulgaris
  source 1..1461: mol_type = genomic DNA
atggcggctgtgtcaactcacccatcaacgagtttaaacagctgtctgaaaaatgagcaaacccaaaaatttatcaagtcccatgctttcttattaaaacctcaaatgttgggttttaatcctagaaaatggaagaatttggagtttgataaactggttgtatctgctagcaatgttgatcagcagggaaatgagagtaatttaccctttaatggcgtctcttcctctactcgatcaaaggcgatgcggcatactatatcagtatttgttggggatgaaagtggaatgataaatcgaattgcaggggtctttgctagaagaggttataatattgaatcccttgctgttgggttgaacgaggacaaagctctcttcaccattgttgtctctggaactgataaggtgttgcaccaagtgatggaacaacttcagaagcttttgaatgttttaaaggttgaagatatatccagggagcctcaggtagaacgtgaattaatgcttgtaaaagttggagctgatcaaagtagccgcggcgagttaatgtggttggtggacatcttccgcgccaaaattgtggacatatctgaagagtatcttacagtagaggtcactggagatccaggaaagatggttgctgtgctaagaaatctaagcaagtttggtatcagagagattgctcgaactggaaagattgctctaagaagggaaaagttgggtgagtctgctcctttctggcgtttttcagcagcttcttatcctgaccttaaagaagctacgctggttgatgccctttctgaagttgcaagacaagcacctgatactggatcatcagatttctccgtcgagggtgatgtttatcctgtggaaccttatgatggtttcgcagttcctcaagtccttgatgctcactggggtgtcttgaatgatgatacaagtgggttccagtcacacactctttcgatgcttgtcaatgacagacccggggtccttaactttgttacaggggcttttgctcgaaggggttataatattcagagtttagctgtgggtcatgcggaaactaagggtctatctcgcatcactactgttgtaactggtacagatgaatcaattagcaaattggtgcagcaaatttataagctggttgatattcatgaggtcaaggatcttacccatttaccgtttgctgagcgagagttgatgttggtaaaggtagctgtgaatactgctgcacgtcgtgaggtccttgacattgccagtatttttcgagctaaagctgttgatgtatctgatcataccattacacttgagctcactggagatgtgaataagatggttgcactccaaagattgttggaaccctatggaatctgtgaggttgcacgaacaggacgtgtggctttggctagagagtcaggtgtggattcaagatatcttcgtggatattcctttcccgtctaa

SEQ ID NO: 11 - protein, 477 aa, Beta vulgaris
MAAVSFNNNGGKIGTLCSKPEFVCGFLRKLDFGAQTTVMAKPMSKISKLKAMEVSSNTSVNAVPVSAHPKVMRHTISVFVGDESGIINRIAGVISRRGYNIESLAVGLNKDKALFTIVVCGTDKVLRQVMEQLNKLVSVLKVEDLSREPQVERELMLVKLNADANTHTEIKWLVEIFRAKIVDVSDSLVTVEVTGDPGKMAAVLRNFSKFGIKEVARTGKIALRRERMGETAPFWRFSAASYPDLEEKAVNTLVESTKRSINGDPGSSSTGDVYPVEPYDNPMVNQVLDAHWGVLHDGDSSGLRSHTLSMLVNNVSGVLNTVTGVISRRGYNIQSLAVGPAEKEGLSRITTVVPGNDESIGKLVQQLSKLVDLHEIQDLTHLPFAERELMLIKVAANTSARRDVLDIANIFRAKAVDVSDHTITLQLSGDLDKMVALQRLLEPYGICEVARTGRVALTRESRVDSALLRGYALPLSE

SEQ ID NO: 12 - DNA, 1434 bp, Beta vulgaris
  source 1..1434: mol_type = genomic DNA
atggcggcggtgtccttcaacaacaatggcggaaagattggaactttgtgttctaaacccgaatttgtttgtgggtttctcagaaaattggattttggtgctcaaacaacagtaatggcaaaacccatgtccaaaatttcaaagttgaaggcaatggaggtttcttcaaatacttcagtaaatgctgtgcctgtttcagctcatcccaaggtgatgcgccacacaatttcagtgtttgttggagatgaaagtgggatcatcaataggattgcaggtgttatatctagaagaggttacaacatagagtctttggctgttggtttaaacaaagataaggctcttttcactatagtagtatgtgggactgataaggtgttgcgccaagtaatggagcaacttaacaagctcgtgagtgtcttgaaggttgaagatctgtcgagagagccacaagtggaacgtgaattgatgcttgtgaagctcaatgctgatgcaaatacccataccgagataaagtggttagtggaaatcttcagggcaaagattgtggatgtctcagacagcttggtcactgttgaggtgactggggatcctggaaagatggctgctgtcctgagaaactttagcaagtttggaattaaagaagttgcaaggacaggaaagatcgctctaaggcgggaaaggatgggtgagacagctcctttttggagattctctgctgcttcttatccagatctagaagagaaggctgttaatactcttgtcgagtctacaaaaagaagtatcaatggtgaccctggctcatcatcaactggtgatgtttatcccgtggaaccctatgataatcccatggtaaatcaagtactcgatgctcactggggtgttcttcatgatggtgattcaagtggactccgatcacacactctgtccatgcttgtgaataatgtttccggggttctaaatactgttacaggagtaatttctcgtaggggttataacattcagagtcttgctgttggcccggctgaaaaggagggtctttctcgtatcacaactgtggttcctggaaatgacgagtcaattggaaagttggtacagcaacttagcaagttagtggaccttcatgagattcaggatcttactcacctgccatttgcggaacgagagcttatgttgatcaaagtagctgcaaatacgtctgccaggagagatgttcttgatattgccaatatttttcgtgcaaaagctgtagatgtttccgatcataccataaccttgcagcttagtggcgatctagacaaaatggttgcgctacagagattattggaaccttatggcatatgtgaggtagcgcggactggaagagttgcactgactagagagtctcgagtagattccgcattattgcgaggatatgctcttcccttgtctgagtaa

SEQ ID NO: 13 - protein, 486 aa, Beta vulgaris; identical to SEQ ID NO: 9

SEQ ID NO: 14 - DNA, 1460 bp, Beta vulgaris
  source 1..1460: mol_type = genomic DNA
atggcggctgtgtcaactcacccatcaacgagtttaaacagctgtctgaaaaatgagcaaacccaaaaatttatcaagtcccatgctttcttattaaaacctcaaatgttgggttttaatcctagaaaatggaagaatttggagtttgataaactggttgtatctgctagcaatgttgatcagcagggaaatgagagtaatttaccctttaatggcgtctcttcctctactcgatcaaaggcgatgcggcatactatatcagtatttgttggggatgaaagtggaatgataaatcgaattgcaggggtctttgctagaagaggttataatattgaatcccttgctgttgggttgaacgaggacaaagctctcttcaccattgttgtctctggaactgataaggtgttgcaccaagtgatggaacaacttcagaagcttttgaatgttttaaaggttgaagatatatccagggagcctcaggtagaacgtgaattaatgcttgtaaaagttggagctgatcaaagtagccgcggcgagttaatgtggttggtggacatcttccgcgccaaaattgtggacatatctgaagagtatcttacagtagaggtcactggagatccaggaaagatggttgctgtgctaagaaatctaagcaagtttggtatcagagagattgctcgaactggaaagattgctctaagaagggaaaagttgggtgagtctgctcctttctggcgtttttcagcagcttcttatcctgaccttaaagaagctacgctggttgatgccctttctgaagttgcaagacaagcacctgatactggatcatcagatttctccgtcgagggtgatgtttatcctgtggaaccttatgatggtttcgcagttcctcaagtccttgatgctcactggggtgtcttgaatgatgatacaagtgggttccagtcacacactctttcgatgcttgtcaatgacagacccggggtccttaactttgttacaggggcttttgctcgaaggggttataatattcagagtttagctgtgggtcatgcggaaactaagggtctatctcgcatcactactgttgtaactggtacagatgaatcaattagcaaattggtgcagcaaatttataagctggttgatattcatgaggtcaaggatcttacccatttaccgtttgctgagcgagagttgtgttggtaaaggtagctgtgaatactgctgcacgtcgtgaggtccttgacattgccagtatttttcgagctaaagctgttgatgtatctgatcataccattacacttgagctcactggagatgtgaataagatggttgcactccaaagattgttggaaccctatggaatctgtgaggttgcacgaacaggacgtgtggctttggctagagagtcaggtgtggattcaagatatcttcgtggatattcctttcccgtctaa

SEQ ID NO: 15 - protein, 477 aa, Beta vulgaris
MAAVSFNNNGGKIGTLCSKPEFVCGFLRKLDFGAQTTVMAKPMSKISKLKAMEVSSNTSVNAVPVSAHPKVMRHTISVFVGDESGIINRIAGVISRRGYNIESLAVGLNKDKALFTIVVCGTDKVLRQVMEQLNKLVSVLKVEDLSREPQVERELMLVKLNADANTHTEIKWLVEIFRAKIVDVSDSLVTVEVTGDPGKMAAVLRNFSKFGIKEVARTGKIALRRERMGETAPFWRFSAASYPDLEEKAVNTLVESTKRSINGDPGSSSTGDVYPVEPYDNPMVNQVLDAHWGVLHDGDSSGLRSHTLSMLVNNVSGVLNTVTGVISRRGYNIQSLAVGPAEKEGLSRITTVVPGNDESIGKLVQQLSKLVDLHEIQDLTHLPFAERELMLIKVAANTSARRDVLDIANIFRAKAVDVSDHTITLQLSGDLDKMVALQRLLEPYGICEVARTGRVALSRESRVDSALLRGYALPLSE

SEQ ID NO: 16 - DNA, 1434 bp, Beta vulgaris
  source 1..1434: mol_type = genomic DNA
atggcggcggtgtccttcaacaacaatggcggaaagattggaactttgtgttctaaacccgaatttgtttgtgggtttcttagaaaattggattttggtgctcaaacaacagtaatggcaaaacccatgtccaaaatttcaaagttgaaggcaatggaggtttcttcaaatacttcagtaaatgctgtgcctgtttcagctcatcccaaggtgatgcgccacacaatttcagtgtttgtcggagatgaaagtgggatcatcaataggattgcaggtgttatatctagaagaggttacaacatagagtctttggctgttggtttaaacaaagataaggctcttttcactatagtagtatgtgggactgataaggtgttgcgccaagtaatggagcaacttaacaagctcgtgagtgtcttgaaggttgaagatctgtcgcgagagccacaagtggaacgtgaattgatgcttgtgaagctcaatgctgatgcaaatacccataccgagataaagtggttagtggaaatcttcagggcaaagattgtggatgtctcagacagcttggtcactgttgaggtgactggggatcctggaaagatggctgctgtcctgagaaattttagcaagtttggaattaaagaagttgcaagaacaggaaagattgctctaaggcgggaaaggatgggtgagacagctcctttttggagattctctgctgcttcttatccagatctagaagagaaggctgttaatactcttgttgagtctacaaaaagaagtatcaatggtgaccctggctcgtcatcaactggtgatgtttatcctgtggagccctatgataatcccatggtaaatcaagtactcgatgctcactggggtgttcttcatgatggtgattcaagtggactccgatcacacactctgtccatgcttgtgaataatgtctccggggttctaaatactgttacaggagtaatttctcgtaggggttataacattcagagtcttgctgttggcccggctgaaaaggagggtctttctcgtatcacaactgtggttcctggaaatgacgagtcaattggaaagttggtacagcaacttagcaagttagtggaccttcatgagattcaggatcttactcacctgccatttgcggaacgagagcttatgttgatcaaagtagctgcaaatacgtctgccaggagagatgttcttgatattgccaatatttttcgtgcaaaagctgtagatgtttccgatcataccataaccttgcagcttagtggcgatctagacaaaatggtcgcgctacagagattattggaaccttatggcatatgtgaggtagcgcggactggaagagttgcactgagtagagagtctcgagtagattccgcattattgcgaggatatgctcttcccttgtccgagtaa

SEQ ID NO: 17 - protein, 486 aa, Beta vulgaris; identical to SEQ ID NO: 9

SEQ ID NO: 18 - DNA, 1461 bp, Beta vulgaris
  source 1..1461: mol_type = genomic DNA
atggcggctgtgtcaactcacccatcaacgagtttaaacagctgtctgaaaaatgagcaaacccaaaaatttatcaagtcccatgctttcttattaaaacctcaaatgttgggttttaatcctagaaaatggaagaatttggagtttgataaactggttgtatctgctagcaatgttgatcagcagggaaatgagagtaatttaccctttaatggcgtctcttcctctactcgatcaaaggcgatgcggcatactatatcagtatttgttggggatgaaagtggaatgataaatcgaattgcaggggtctttgctagaagaggttataatattgaatcccttgctgttgggttgaacgaggacaaagctctcttcaccattgttgtctctggaactgataaggtgttgcaccaagtgatggaacaacttcagaagcttttgaatgttttaaaggttgaagatatatccagggagcctcaggtagaacgtgaattaatgcttgtaaaagttggagctgatcaaagtagccgcggcgagttaatgtggttggtggacatcttccgcgccaaaattgtggacatatctgaagagtatcttacagtagaggtcactggagatccaggaaagatggttgctgtgctaagaaatctaagcaagtttggtatcagagagattgctcgaactggaaagattgctctaagaagggaaaagttgggtgagtctgctcctttctggcgtttttcagcagcttcttatcctgaccttaaagaagctacgctggttgatgccctttctgaagttgcaagacaagcacctgatactggatcatcagatttctccgtcgagggtgatgtttatcctgtggaaccttatgatggtttcgcagttcctcaagtccttgatgctcactggggtgtcttgaatgatgatacaagtgggttccagtcacacactctttcgatgcttgtcaatgacagacccggggtccttaactttgttacaggggcttttgctcgaaggggttataatattcagagtttagctgtgggtcatgcggaaactaagggtctatctcgcatcactactgttgtaactggtacagatgaatcaattagcaaattggtgcagcaaatttataagctggttgatattcatgaggtcaaggatcttacccatttaccgtttgctgagcgagagttgatgttggtaaaggtagctgtgaatactgctgcacgtcgtgaggtccttgacattgccagtatttttcgagctaaagctgttgatgtatctgatcataccattacacttgagctcactggagatgtgaataagatggttgcactccaaagattgttggaaccctatggaatctgtgaggttgcacgaacaggacgtgtggctttggctagagagtcaggtgtggattcaagatatcttcgtggatattcctttcctgtctaa

SEQ ID NO: 19 - protein, 477 aa, Beta vulgaris; identical to SEQ ID NO: 15

SEQ ID NO: 20 - DNA, 1434 bp, Beta vulgaris; identical to SEQ ID NO: 16
  source 1..1434: mol_type = genomic DNA

SEQ ID NO: 21 - protein, 486 aa, Beta vulgaris
MAAVSTHPSTSLNSCLKNEQTQKFIKSHAFLLKPQMLGFNPRKWKNLEFDKLVVSASNVDHQGNESNLPFNGVSSSTRSKAMRHTISVFVGDESGMINRIAGVFARRGYNIESLAVGLNEDKALFTIVVSGTDKVLHQVMEQLQKLLNVLKVEDISREPQVERELMLVKVGADQSSRGELMWLVDIFRAKIVDISEEYLTVEVTGDPGKMVAVLRNLSKFGIREIARTGKIALRREKLGESAPFWRFSAASYPDLKEATLVDALSEVARQAPDTKSSDFSVEGDVYPVEPYDGFAVPQVLDAHWGVLNDDTSGFQSHTLSMLVNDRPGVLNFVTGAFARRGYNIQSLAVGHAETKGLSRITTVVTGTDESISKLVQQIYKLVDIHEVKDLTHLPFAERELMLVKVAVNTAARREVLDIASIFRAKAVDVSDHTITLELTGDVNKMVALQRLLEPYGICEVARTGRVALARESGVDSRYLRGYSFPV

SEQ ID NO: 22 - DNA, 1461 bp, Beta vulgaris
  source 1..1461: mol_type = genomic DNA
atggcggctgtgtcaactcacccatcaacgagtttaaacagctgtctgaaaaatgagcaaacccaaaaatttatcaagtcccatgctttcttattaaaacctcaaatgttgggttttaatcctagaaaatggaagaatttggagtttgataaactggttgtatctgctagcaatgttgatcatcagggaaatgagagtaatttaccctttaatggcgtttcttcctctactcgatcaaaggcgatgcggcatactatatcagtatttgttggggatgaaagtggaatgataaatcgaattgcaggggtctttgctagaagaggttataatattgaatcccttgctgttgggttgaacgaggacaaagctctcttcaccattgttgtctctggaactgataaggtgttgcaccaagtgatggaacaacttcagaagcttttgaatgttttaaaggttgaagatatatccagggagcctcaggtagaacgtgaattaatgcttgtaaaagttggagctgatcaaagtagccgcggtgagttaatgtggttggtggacatcttccgcgccaaaattgtggacatatctgaagagtatcttacagtagaggtcactggagatccaggaaagatggttgctgtgctaagaaatctaagcaagtttggtatcagagagattgctcgaactggaaagattgctctaagaagagaaaagttgggtgagtctgctcctttctggcgtttttcagcagcttcttatcctgaccttaaagaagctacgctggttgatgccctttctgaagttgcaagacaagcacctgatactaaatcatcagatttctccgtcgagggtgatgtttatcctgtggaaccttatgatggtttcgcagttcctcaagtccttgatgctcactggggtgtcttgaatgatgatacaagtgggttccagtcacacactctttcgatgcttgtcaatgacagacccggggtccttaactttgttacaggggcttttgctcgaaggggttataatattcagagtttagctgtgggtcatgcggaaactaagggtctatctcgcatcactactgttgtaactggtacagatgaatcaattagcaaattggtgcagcaaatttataagctggttgatattcatgaggtcaaggatcttacccatttaccgtttgctgagcgagagttgatgttggtaaaggtagctgtgaatactgctgcacgtcgtgaggtccttgacattgccagtatttttcgagctaaagctgttgatgtatctgatcataccattacacttgagctcactggagatgtgaataagatggttgcactccaaagattgttggaaccctatggaatctgcgaggttgcacgaacaggacgtgtggctttggctagagagtcaggtgtggactcaagatatcttcgtggatattcctttcctgtctaa

SEQ ID NO: 23 - protein, 477 aa, Beta vulgaris; identical to SEQ ID NO: 11

SEQ ID NO: 24 - DNA, 1434 bp, Beta vulgaris; identical to SEQ ID NO: 12
  source 1..1434: mol_type = genomic DNA

SEQ ID NO: 25 - protein, 486 aa, Beta vulgaris; identical to SEQ ID NO: 21

SEQ ID NO: 26 - DNA, 1461 bp, Beta vulgaris; identical to SEQ ID NO: 22
  source 1..1461: mol_type = genomic DNA

SEQ ID NO: 27 - protein, 477 aa, Beta vulgaris; identical to SEQ ID NO: 11

SEQ ID NO: 28 - DNA, 1434 bp, Beta vulgaris; identical to SEQ ID NO: 12
  source 1..1434: mol_type = genomic DNA

SEQ ID NO: 29 - protein, 486 aa, Beta vulgaris; identical to SEQ ID NO: 21

SEQ ID NO: 30 - DNA, 1461 bp, Beta vulgaris; identical to SEQ ID NO: 22
  source 1..1461: mol_type = genomic DNA

SEQ ID NO: 31 - protein, 477 aa, Beta vulgaris; identical to SEQ ID NO: 11

SEQ ID NO: 32 - DNA, 1434 bp, Beta vulgaris; identical to SEQ ID NO: 12
  source 1..1434: mol_type = genomic DNA

SEQ ID NO: 33 - DNA, 50 bp, synthetic construct
  misc_feature 1..50: note = Gene marker M1
  source 1..50: mol_type = other DNA
catttaccgtttgctgagcgagagttgatgttggtaaaggtagctgtgaa

SEQ ID NO: 34 - DNA, 50 bp, synthetic construct
  misc_feature 1..50: note = Gene marker M2
  source 1..50: mol_type = other DNA
ggtagggagctacatttgatgcttagttgtatgagttgtgatggcttaaa

SEQ ID NO: 35 - DNA, 50 bp, synthetic construct
  misc_feature 1..50: note = Gene marker M3
  source 1..50: mol_type = other DNA
tactatccctctattacttccgggctaatcgaacgaatttccgaaagggc

SEQ ID NO: 36 - DNA, 50 bp, synthetic construct
  misc_feature 1..50: note = Gene marker M4
  source 1..50: mol_type = other DNA
gagtctcgagtagattccgcattattgcgaggatatgctcttcccttgtc

SEQ ID NO: 37 - DNA, 50 bp, synthetic construct
  misc_feature 1..50: note = Left marker M5
  source 1..50: mol_type = other DNA
tccgaagtagtcgccggaagagatgagagaaaaatagagagagaaataga

SEQ ID NO: 38 - DNA, 50 bp, synthetic construct
  misc_feature 1..50: note = Left marker M6
  source 1..50: mol_type = other DNA
tgaatattctatcaaattatggaatgaggatatttgaatgttggagttgc

SEQ ID NO: 39 - DNA, 50 bp, synthetic construct
  misc_feature 1..50: note = Left marker M7
  source 1..50: mol_type = other DNA
tgactaaatgcaatcatatagtagaaaagatttgaccaataatttctctt

SEQ ID NO: 40 - DNA, 50 bp, synthetic construct
  misc_feature 1..50: note = Left marker M8
  source 1..50: mol_type = other DNA
ttatcaactccctgatcaagtagagacttgtcaatggcagaacgaagatc

SEQ ID NO: 41 - DNA, 50 bp, synthetic construct
  misc_feature 1..50: note = Left marker M9
  source 1..50: mol_type = other DNA
acacgctttacaaactcgggaaacaaatgcaagtaatcactctgaaggaa

SEQ ID NO: 42 - DNA, 50 bp, synthetic construct
  misc_feature 1..50: note = Left marker M10
  source 1..50: mol_type = other DNA
atggtaaacatagctttatcggctgtgcttttgtttgcaattcgcaacag

SEQ ID NO: 43 - DNA, 50 bp, synthetic construct
  misc_feature 1..50: note = Right marker M11
  source 1..50: mol_type = other DNA
agagaacattttgttctatactggactatgctaattctcaatcgcacctg

SEQ ID NO: 44 - DNA, 50 bp, synthetic construct
  misc_feature 1..50: note = Right marker M12
  source 1..50: mol_type = other DNA
atcaagtccagttggtgaagagaaaggattccaatcgctacgaaattgtc

SEQ ID NO: 45 - DNA, 50 bp, synthetic construct
  misc_feature 1..50: note = Right marker M13
  source 1..50: mol_type = other DNA
tatatagatatatattctcctgttataaattataacagcaattagtggaa

SEQ ID NO: 46 - DNA, 50 bp, synthetic construct
  misc_feature 1..50: note = Right marker M14
  source 1..50: mol_type = other DNA
cttccagctctaacaccagccacccaatgagatatgtttcctttccctcg

SEQ ID NO: 47 - DNA, 50 bp, synthetic construct
  misc_feature 1..50: note = Right marker M15
  source 1..50: mol_type = other DNA
ttgtggtgaattcgcaagagaattaatggtttttcgagtatgcagtgact

SEQ ID NO: 48 - DNA, 50 bp, synthetic construct
  misc_feature 1..50: note = Right marker M16
  source 1..50: mol_type = other DNA
tttgttcttttgtgagggagctgaaggaacttcaaacaattgatgctttg